SEQUENCE LISTING (4 sequences)

SEQ ID NO: 1 - protein, 299 aa, Homo sapiens [sequence omitted]

SEQ ID NO: 2 - DNA, 16 bp, Artificial Sequence
  Synthesized
tattaatcgattaatg

SEQ ID NO: 3 - DNA, 30 bp, Artificial Sequence
  Synthesized
aattaagcttctgtaagaatctgagatgcc

SEQ ID NO: 4 - DNA, 310 bp, Artificial Sequence
  Synthesized
gatccgacgccatctctaggcccgcgccggccccctcgcacagacttgtggagaagctcggctactcccctgccccggttaatttgcatataatatttcctagtaactatagaggcttaatgtgcgataaaagacagataatctgttctttttaatactagctacattttacatgataggcttggatttctataagagatacaaatactaattattattttaaaaaacagcacaaaaggaaactcaccctaactgtaagtaattgtgtgttttgagactataaatatcccttggagaaaagccttgtttg

(19) United States
(12) Patent Application Publication
(10) Pub. No.: US 2024/0207449 A1
(43) Pub. Date: Jun. 27, 2024

(54) COMPOSITIONS AND METHODS FOR TREATMENT OF CRX-MEDIATED RETINOPATHIES

(71) Applicant: Washington University, St. Louis, MO (US)

(72) Inventor: Shiming CHEN, St. Louis, MO (US)

(21) Appl. No.: 18/286,205

(22) PCT Filed: Apr. 11, 2022
(86) PCT No.: PCT/US2022/024246

§ 371 Date: Oct. 9, 2023

Related U.S. Application Data
(60) Provisional application No. 63/172,978, filed on Apr. 9, 2021.

(51) Int. Cl. A61K 48/00; A61P 27/02; C12N 15/86

(52) U.S. Cl. CPC A61K 48/0058; A61P 27/02; C12N 15/86; C12N 2750/14122; C12N 2750/14143; C12N 2750/14145; C12N 2750/14171; C12N 2830/006

(57) ABSTRACT

Among the various aspects of the present disclosure is the provision of compositions and methods of making regulatable, adeno-associated viral vectors for the therapeutic expression of CRX and methods of use thereof.

### CROSS REFERENCE TO RELATED APPLICATIONS

This application claims priority of U.S. provisional application No. 63/172,978, filed Apr. 9, 2021, the disclosure of which is hereby incorporated by reference in its entirety.

### GOVERNMENTAL RIGHTS

This invention was made with government support under EY032136 awarded by the National Institutes of Health. The government has certain rights in the invention.

### REFERENCE TO A SEQUENCE LISTING

This application contains a Sequence Listing that has been submitted in ASCII format via EFS-Web and is hereby incorporated by reference in its entirety. The ASCII copy, created on Apr. 11, 2022, is named “Untitled_ST25.txt”, and is 4,096 bytes in size.

### FIELD OF THE TECHNOLOGY

The present disclosure relates to gene therapy for retinal and mutant Cone-Rod Homeobox (CRX)-mediated disorders. In particular, disclosed herein are compositions and methods for treating retinal disease comprising the regulatable expression of the transcription factor Cone-Rod Homeobox (CRX).

### BACKGROUND

Inherited retinal diseases (IRDs) are a major cause of registered and largely incurable blindness worldwide. Mutations in as many as 300 genes can cause IRDs, with approximately 70 genes identified as causative for the most common condition retinitis pigmentosa (RP) (information available on the internet, see for example, sph.uth.edu/retnet/). IRDs are currently the most diverse of the described hereditary conditions in humans. Leber congenital amaurosis (LCA) constitutes a group of early onset blinding diseases (in young children) with at least 25 causal genes that overlap with RP genes. Typically, IRDs are characterized by gradual loss of light sensing photoreceptor cells in the retina at the back of the eye leading to reduced light detection capacity and eventually blindness. The most common modes of inheritance are autosomal recessive, autosomal dominant and X-linked, but digenic and mitochondrial etiology have also been observed. Frequently, patients with not only different mutations in the same gene but also with the exact same mutation demonstrate divergent clinical phenotypes, presenting a challenge to patient counseling and disease management. Many pathological mutations are found in genes that affect photoreceptor-specific functions within the retina (den Hollander et al., Prog Retin Eye Res, 27, 391-419; Wright et al., Nat Rev Genet, 11, 273-284).

Precisely regulated gene expression is essential for photoreceptor development and maintenance. This process is governed by a genetic program centered on the cone-rod homeobox transcription factor CRX. Mutations in the human CRX gene have been associated with dominant retinopathies with a wide-range of phenotypes and ages of onset. CRX gene mutations are responsible for about one-quarter of the cases of a form of the condition called autosomal dominant cone-rod dystrophy. These mutations lead to a reduction in the amount of functional cone-rod homeobox protein that is available to regulate other genes in the retina. However, a poor understanding of the mechanism of each individual mutation has made it difficult to develop treatment strategies.

Therefore, a need in the art exists for compositions and methods which allow for a gene augmentation approach that allows for regulatable and/or targeted therapeutic CRX production.

### SUMMARY

Among the various aspects of the present disclosure are provided compositions and methods of use thereof in gene therapy of CRX mutational disorders.

Another aspect of the present disclosure encompasses a regulatable, recombinant adeno-associated viral vector having a first promoter operably linked to the first nucleic acid sequence, wherein the first nucleic acid sequence encodes a tetracycline-controlled transactivator; and a second promoter comprising a tet-regulated element (TRE), wherein the second promoter is operably linked to the second nucleic acid sequence, wherein the second nucleic acid sequence encodes a CRX protein that provides a therapeutic effect on a CRX-mediated retinopathy.

In some embodiments, the TRE region comprises at least one tetracycline resistance operator binding site and wherein the promoter is a minimal promoter region comprising a TATA box and transcription start site. In some embodiments, the first promoter is a photoreceptor cell specific promoter. In exemplary embodiments, the photoreceptor cell specific promoter is selected from a GRK promoter, a CRX promoter, a CrxdEN promoter and a Gnbq501/3 promoter. In some embodiments, the second promoter is regulatable by tetracycline or doxycycline. In some embodiments, the second promoter is “OFF” regulatable, wherein the expression is about 5% or less compared to the expression without tetracycline or doxycycline. In some embodiments, the promoter system is “OFF” regulatable, wherein the expression is about 1% or less compared to the expression without tetracycline or doxycycline.

In some embodiments, the vector is an AAV serotype. In some embodiments, the adeno-associated viral vector is an AAV2 serotype. In some embodiments, the adeno-associated viral vector is an AAV5 serotype. In some embodiments, the adeno-associated viral vector is an AAV2/5 serotype.

Another aspect of the present disclosure encompasses methods of delivering a regulatable, adeno-associated viral vector to a photoreceptor cell in a subject with a CRX-mediated retinopathy, the method generally includes providing a recombinant adeno-associated viral (rAAV) vector, the vector having a first promoter operably linked to the first nucleic acid sequence, wherein the first nucleic acid sequence encodes a tetracycline-controlled transactivator; and a second promoter comprising a tet-regulated element (TRE), wherein the second promoter is operably linked to the second nucleic acid sequence, wherein the second nucleic acid sequence encodes a CRX protein; and administering the rAAV vector to the photoreceptor cell.

In some embodiments, the TRE region comprises at least one tetracycline resistance operator binding site and wherein the promoter is a minimal promoter region comprising a TATA box and transcription start site. In some embodiments, the first promoter is a photoreceptor cell specific promoter. In some embodiments, the photoreceptor cell specific promoter is selected from a GRK promoter, a CRX promoter, a CrxdEN promoter or a Gnbq501/3 promoter. In some embodiments, the second promoter is regulatable by tetracycline or doxycycline.

In some embodiments, the method further comprises administering tetracycline or doxycycline after the administration of the rAAV vector to reduce expression of CRX in the photoreceptor cell. In some embodiments, the expression of CRX is about 5% or less compared to the expression without tetracycline or doxycycline.

Another aspect of the present disclosure encompasses methods of treating a subject having or suspected of having a CRX-mediated retinopathy, the method generally includes providing a recombinant adeno-associated viral (rAAV) vector, the vector comprising: a first promoter operably linked to the first nucleic acid sequence, wherein the first nucleic acid sequence encodes a tetracycline-controlled transactivator; and a second promoter comprising a tet-regulated element (TRE), wherein the second promoter is operably linked to the second nucleic acid sequence, wherein the second nucleic acid sequence encodes a CRX protein; and administering the rAAV vector to the subject.

In some embodiments, the TRE region comprises at least one tetracycline resistance operator binding site and wherein the promoter is a minimal promoter region comprising a TATA box and transcription start site. In some embodiments, the first promoter is a photoreceptor cell specific promoter. In some embodiments, the photoreceptor cell specific promoter is selected from a GRK promoter, a CRX promoter, a CrxdEN promoter or a Gnbq501/3 promoter. In some embodiments, the second promoter is regulatable by tetracycline or doxycycline.

In some embodiments, the method further comprises administering tetracycline or doxycycline to the subject after the administration of the rAAV vector to reduce expression of CRX in a photoreceptor cell of the subject. In some embodiments, the expression of CRX is about 5% or less compared to the expression without tetracycline or doxycycline.

Another aspect of the present disclosure encompasses methods of treating a cone rod homeobox transcription factor (CRX) autosomal dominant retinopathy in a subject, the method generally includes administering to the subject an effective amount of a nucleic acid molecule comprising a first photoreceptor cell specific promoter operably linked to a nucleic acid molecule encoding a CRX protein and further comprising a second photoreceptor cell specific promoter operably linked to a nucleic acid molecule encoding a shRNA specific for a mutant CRX mRNA, thereby treating the CRX autosomal dominant retinopathy in the subject.

In some embodiments, the CRX autosomal dominant retinopathy is Leber congenital amaurosis (LCA), retinitis pigmentosa, or cone rod dystrophy. In a specific embodiment, the CRX autosomal dominant retinopathy is the LCA.

In some embodiments, the methods include administering to the subject a viral vector comprising the nucleic acid molecule. In some embodiments, the viral vector is a lentivirus vector or an adeno-associated vims (AAV) vector. In some embodiments, the viral vector is the AAV vector, and wherein the AAV vector is an AAV2, AAV5, AAV8 virus vector or any combination thereof. In a specific embodiment, the AAV vector is AAV2/5.

In some embodiments, the methods include administering to the subject a nanoparticle or a dendrimer comprising the nucleic acid molecule.

In some embodiments, the first promoter is GnbQ5 and the second promoter is constitutively active. In a specific embodiment, the second promoter is U6 promoter.

In some embodiments, the nucleic acid molecule is administered intravitreally, subretinally or to the retina of the subject. In some embodiments, the subject is a human. In some embodiments, the method increases cone Arrestin, Rhodopsin, and/or cone Opsin expression in the retina of the subject. In some embodiments, the method includes selecting the subject with the CRX autosomal dominant retinopathy.

Another aspect of the present disclosure encompasses compositions comprising an effective amount of a nucleic acid molecule comprising a first photoreceptor cell specific promoter operably linked to a nucleic acid molecule encoding a CRX protein and further comprising a second photoreceptor cell specific promoter operably linked to a nucleic acid molecule encoding a shRNA specific for a mutant CRX mRNA for use in treating a cone rod homeobox transcription factor (CRX) autosomal dominant retinopathy in a subject. In some embodiments, the CRX autosomal dominant retinopathy is Leber congenital amaurosis (LCA), retinitis pigmentosa, or cone rod dystrophy. In a specific embodiment, the CRX autosomal dominant retinopathy is LCA.

In some embodiments, the composition comprises a viral vector, wherein the viral vector comprises the nucleic acid molecule comprising a first photoreceptor cell specific promoter operably linked to a nucleic acid molecule encoding a CRX protein and further comprising a second photoreceptor cell specific promoter operably linked to a nucleic acid molecule encoding a shRNA specific for a mutant CRX mRNA. In some embodiments, the viral vector is a lentiviral vector or an adeno-associated vims (AAV) vector. In some embodiments, the viral vector is the AAV vector, and wherein the AAV vector is an AAV2, AAV5, AAV8 virus vector or any combination thereof. In some embodiments, the AAV vector is AAV2/5.

In some embodiments, the composition comprises a nanoparticle or a dendrimer, wherein the nanoparticle or dendrimer comprising the nucleic acid molecule.

In some embodiments, the first promoter is GnbQ50 and the second promoter is constitutively active. In a specific embodiment, the second promoter is U6 promoter.

In some embodiments, the composition is formulated for retinal or subretinal administration.

In some embodiments, the subject is a human wherein treating a CRX autosomal dominant retinopathy comprises increasing cone Arrestin, Rhodopsin, and/or cone Opsin expression in the retina of the subject.

Another aspect of the present disclosure encompasses a nucleic acid molecule comprising a first photoreceptor cell specific promoter operably linked to a nucleic acid molecule encoding a CRX protein and further comprising a second photoreceptor cell specific promoter operably linked to a nucleic acid molecule encoding a shRNA specific for a mutant CRX mRNA. In some embodiments, the nucleic acid molecule is in a vector wherein the vector is a viral vector. In some embodiments, the viral vector is an AAV vector.

Another aspect of the present disclosure encompasses compositions comprising an effective amount of a nucleic acid molecule comprising a photoreceptor cell specific promoter operably linked to a nucleic acid molecule encoding a CRX, wherein the promoter is not a CRX promoter or a promoter that is trans activated by wild-type CRX for use in treating a cone rod homeobox transcription factor (CRX) autosomal dominant retinopathy in a subject. In some embodiments, the CRX autosomal dominant retinopathy is Leber congenital amaurosis (LCA), retinitis pigmentosa, or cone rod dystrophy. In a specific embodiment, the CRX autosomal dominant retinopathy is LCA.

In some embodiments, the composition comprises a viral vector, wherein the viral vector comprises the nucleic acid molecule comprising a first photoreceptor cell specific promoter operably linked to a nucleic acid molecule encoding a CRX protein and further comprising a second photoreceptor cell specific promoter operably linked to a nucleic acid molecule encoding a shRNA specific for a mutant CRX mRNA. In some embodiments, the viral vector is a lentiviral vector or an adeno-associated vims (AAV) vector. In some embodiments, the viral vector is the AAV vector, and wherein the AAV vector is an AAV2, AAV5, AAV8 virus vector or any combination thereof. In some embodiments, the AAV vector is AAV2/5.

In some embodiments, the composition comprises a nanoparticle or a dendrimer, wherein the nanoparticle or dendrimer comprising the nucleic acid molecule.

In some embodiments, the first promoter is GnbQ50 and the second promoter is constitutively active. In a specific embodiment, the second promoter is U6 promoter.

In some embodiments, the composition is formulated for retinal or subretinal administration.

In some embodiments, the subject is a human wherein treating a CRX autosomal dominant retinopathy comprises increasing cone Arrestin, Rhodopsin, and/or cone Opsin expression in the retina of the subject.

Another aspect of the present disclosure encompasses a nucleic acid molecule comprising a photoreceptor cell specific promoter operably linked to a nucleic acid molecule encoding a CRX, wherein the promoter is not a CRX promoter or a promoter that is trans activated by wild-type CRX. In some embodiments, the nucleic acid molecule is in a vector wherein the vector is a viral vector. In some embodiments, the viral vector is an AAV vector.

These and other aspects and iterations of the invention are described more thoroughly below.

### BRIEF DESCRIPTION OF THE FIGURES

The application file contains at least one drawing executed in color. Copies of this patent application publication with color drawing(s) will be provided by the Office upon request and payment of the necessary fee.

FIG. 1A-1D show AAV-TetOff-CRX vectors to express regulatable CRX in photoreceptors (PhR). FIG. 1A is a diagram of the regulatable photoreceptor-specific AAV-TetOff-CRX vectors. Each vector contains two genes, a photoreceptor promoter-driven tTA activator (a) and a tTA-responsive (TRE) promoter-driven hCRX (FIG. 1B). FIG. 1B is a table which lists four candidate PhR promoters to be used to build AAV-TetOff-CRX vectors. (C&D) preliminary results of ex vivo tests (electroporation to P0 Crx−/− retinal explants) for two such vectors (highlighted) in the Table. FIG. 1C shows FLAG immunostaining (green) of P8 Crx−/− retinal explants that received the indicated vectors at P0 by electroporation. The results indicate that these two vectors can deliver CRX expression into Crx−/− photoreceptor cells. FIG. 1D shows qRT-PCR for ectopically expressed hCRX mRNA in P8 retinal explants treated with Dox (1 μg/mL) at P6 vs untreated (−Dox) controls (set as “1.0”). Error bars are based on technical replicates. The results suggest that the amount of CRX produced by each vector can be tuned down by Dox.

FIG. 2 is the map and sequence information of rAAV-pGRK-TetOff-CRX (7547 bp) and rAAV-pGnb3Q50-TetOff-CRX (8053 bp). These two vectors have been tested ex vivo and packed into AAV2/5 viral particles. Control AAV2/5 vectors where dsRed replaced hCRX have also been made. Two other CRX vectors driven by a pCrx promoter are also made.

FIG. 3 is a diagram of the Dox-inducible TetOn-hCRX transgenic system. Administration of doxycycline (Dox) triggers a conformation change of the reverse tetracycline-controlled transcriptional activator (rtTA) allowing it to bind and activate tetracycline-responsive elements (TREs) to drive expression of a FLAG-tagged human CRX (TRE-hCRX inserted at the H11 locus). To achieve a photoreceptor-specific rtTA expression, two more transgenes have been introduced: One contains a loxP-Stop-LoxP cassette in the rtTA promoter (Rosa26: pCAG-LSL-rtTA), which makes rtTA only in the presence of Cre recombinase; the other contains a photoreceptor promoter-driven Cre (pNrI-Cre, or pCrxCre). These three transgenes together constitute the TetOn-CRX system for photoreceptor-specific and Dox-inducible CRX expression.

FIG. 4A-4E show studies of Dox-induced expression of hCRX and Rho in Crx−/− retinae. FIG. 4A is the experimental scheme for Dox induction [Intraperitoneal (IP) injection or Dox chow (200 mg/kg)] and harvest. FIG. 4B shows qRT-PCR assays for hCRX and Rho following the indicated IP treatments. WT values provided for reference. FIG. 4C shows qRT-PCR assays for hCRX and Rho following the indicated IP treatments. WT values provided for reference. FIG. 4D shows H&E stained retinal sections of P21+14/20 mg/mL Dox treated & control mice. FIG. 4E shows dox chow-induced expression of Rho protein (a CRX target gene) (green) and preserved ONL thickness (blue).

FIG. 5A-5C shows induced TetON-hCRX expression improved the photoreceptor morphology and Rho expression in CrxE168d2/+ mouse retina. hCRX expression was induced at P21 by feeding the experimental mice with Dox chow. The retina was harvested at P35 to access Rhodopsin (Rho) expression and the thickness of the rod outer segments (OS). FIG. 5A is immunostaining for Rho (red), showing rod outer segments (OS) that are positive for Rho. FIG. 5B show quantification of changes in the OS thickness (double-head arrows in A) for treated (+hCRX) and untreated (−hCRX) mice. FIG. 5C show qRT-PCR analysis of expressive levels of induced hCRX and Rho mRNA. These results show that Rho expression and the rod OS thickness were significantly improved in hCRX-induced retina as compared to the untreated control retina (−hCRX).

FIG. 6 shows phenotypic improvements in treated E168d2/+ mice. Treated retinas show improved photoreceptor (ONL cell) survival and increased thickness of the outer segments (OS) that are essential for visual function.

FIG. 7 shows ectopic hCRX expression in CrxE168d2/+ improved rod/cone gene expression and thickness of photoreceptor outer segments (OS). TetON-hCRX was turned on in Crx-E168d2/+ mouse retina by +Dox food at P21. The retina was harvested at 3 mon to assess the protein expression of Rhodopsin (Rho) and cone arrestin (ARR3) and changes of thickness of the outer segments (OS). The ectopic hCRX expression improved Rho expression and rod OS thickness and ARR3 expression in cones, as compared to the untreated control retina (−hCRX).

FIG. 8 shows functional improvements in treated E168d2/+ mice using electroretinogram measures. hCRX-treated animals (Cre+) showed improved rod (Dark) and cone (Light)-driven ERG responses, even when the treatment started late at P21. Excitingly, PLR and Optokinetics visual behavior tests also showed that the treatment greatly improved visual acuity—The cone function is significantly improved in treated retina at 3 mon of age in Crx-E168d2/+ mouse model.

FIG. 9 is a schematic showing visual behavior test I: pupillary light reflex (PLR). Reflexive constriction of the pupil to bright light. In the dark, the pupil dilates to allow as much light as possible to enter the eye. In bright light, the pupil constricts to protect the retina from damaging light. Initiated by photoreceptor and photosensitive retinal ganglion cells and requires the function of entire visual pathway: Retina→Brain→Ocular motor nerve→Iris muscle.

FIG. 10 shows methods for measuring Pupillary Light Reflex (PLR) in mice.

FIG. 11 shows hCRX-treated E168d2/+ mice show improved PLR. Note that the treated eyes have recovered PLR responses similar to the level of normal eyes.

FIG. 12 shows visual behavior test II: Optokinetic Reflex (OKR) to measure visual acuity. OKR->reflexive ability of the eye to track drifting bars. Starts with slow tracking movement in direction of bar movement. Followed by quick saccade back to track the next bar. Both rods and cones drive OKR responses. Can count the number of eye-tracking movements (ETMs, *)/60 s of the stimulus Can change size of the bars to assess visual acuity. Maximum visual acuity associated with maximum ETMs.

FIG. 13 shows hCRX-treated E168d2/+ mice show improved OKR responses. Note that the treated eyes have recovered OKR responses similar to the level of normal eyes, suggesting greatly improved cone function and visual acuity.

FIG. 14 shows AAV2/5-GnbQ50TetOff-hCRX can transduce P0 E168d2/+ retina. 1×1012 PFU (2 uL) of a viral prep containing AAV2/5-GnbQ50TetOff-hCRX (therapy vector) and AAV2/5-GnbQ50 tdTomato (for marking the transduced area) were injected into the subretinal space of a PO E168d2/+ mouse. The AAV transduced area (tdTomato red fluorescence) and expression of cone arrestin (immunostaining, green) were assessed at 1 month using whole-mount retina preps. Panels were from two AAV injected E168d2/+ retinal samples with marked transduced and untransduced areas. These results demonstrate that 1) The AAV2/5 virus is capable of transducing P0 mutant retina; 2) The pGnbQ50 promoter can actively drive the expression of a target gene in mutant retinas carrying a dominant CRX mutation; and 3) Ectopic hCRX expression delivered by AAV2/5 can rescue defective cones to express an essential cone gene.

FIG. 15 shows a dual function AAV design to treat more severe disease.

FIG. 16 shows a dual function vector (knockdown+gene augmentation) rescued Rho expression in Crx-E168d2/d2 mouse retina more effectively than a single function vector (augmentation only)

FIG. 17 shows the dual function vector rescued RHO expression more effectively than the single function vector by immunohistochemistry. Immunostaining of RHO (A&B) and FLAG (hCRX, C&D) on cross sections of P14 E168d2/d2 retinal explants that received the indicated vector at PO. Note that both vectors expressed hCRX (FLAG+, C&D), but only the dual function vector with mCrxShRNA effectively rescued Rho expression. Thus, the knockdown plus gene augmentation strategy has a big advantage over gene augmentation alone to treat diseased retinae.

FIG. 18 shows the vector map for pAAV-mCrx-shRNA-Gnb3Q50.

### DETAILED DESCRIPTION

The present disclosure is based, in part, on the discovery that modulating the mutant/WT CRX ratio is a critical aspect for rescuing photoreceptor cell phenotype and function. For example, low expression levels of the therapeutic CRX relative to mutant CRX are unable to overcome the negative effects of the CRX mutant protein, whereas on the other hand, retinae which already overproduce the mutant CRX protein are sensitive to overdose toxicity when a therapeutic CRX is expressed at high levels. Thus, promoter selection in addition to the ability to regulate mutant/WT CRX ratios are important aspects of the present disclosure.

The present disclosure provides compositions and methods for targeting the expression of a heterologous CRX in cells of the retina. In particular, retinal cells are modified with an nucleic acid sequence useful for the regulatable expression of a heterologous CRX gene and optionally expression of a shRNA which specifically binds to endogenous CRX RNA. In some embodiments, the nucleic acid sequence comprises regulatory sequences (e.g. cell type and expression level specific promoters and tetracycline-response elements (TREs)) which are operably linked to a nucleic acid sequence encoding a cone-rod homeobox protein (CRX). As described herein, photoreceptor deficits caused by recessive or dominant CRX mutations can be rescued by introducing the appropriate amount of normal CRX. Antimorphic CRX mutations cause dominant disease by interfering with the function of normal CRX allele or other TFs in CRX-GRN. The Class Ill mouse model E168d2/+ demonstrated that the protein ratio of WT/mutant allele directly correlates with phenotype severity and age of onset. A small increase in the WT/mutant ratio in E168d2neo/+ mice resulted in a much-improved phenotype. Furthermore, as shown herein E168d2/+ retinae that received transgenic hCRX expression at P21 have significantly improved Rho expression and rod outer segment (OS) formation at P35, thus, supporting the finding that raising of the WT/mutant ratio by expressing an ectopic normal CRX has a beneficial effects on photoreceptor integrity in dominant CRX disease. No treatment is currently available for CRX disease.

In some aspects, the nucleic acid sequence as described herein can be included in a plasmid, viral vector, or gene editing construct. Thus, a composition of the present disclosure provides a vector for the regulatable expression of CRX in a photoreceptor cell. A vector of the disclosure includes vectors derived from a virus such as an adeno-associated virus (AAV). A composition of the disclosure may optionally comprise one or more additional drug or therapeutically active agent in addition to the disclosed nucleic acid sequence. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, salts (substances of the present invention may themselves be provided in the form of a pharmaceutically acceptable salt), buffers, coating agents, or antioxidants.

Other aspects and iterations of the invention are described more thoroughly below.

### I. Compositions

Provided herein are compositions for use in gene therapy. In one aspect, the compositions include a nucleic acid molecule comprising a photoreceptor specific promoter operably linked to a nucleic acid molecule encoding the cone-rod homeobox protein (CRX) protein. In some embodiments, the nucleic acid molecule further comprises a second promoter operably linked to a nucleic acid molecule encoding an inhibitory RNA molecule (e.g., shRNA or siRNA) which specifically targets mutant CRX mRNA but not wild-type CRX mRNA.

In another aspect, compositions of the disclosure include a nucleic acid molecule comprising a first promoter operably linked to a first nucleic acid encoding a tetracycline-controlled transactivator (tTA); and a second promoter which is a tTA-responsive (TRE) promoter operably linked to a second nucleic acid sequence encoding the CRX. In some embodiments, the first promoter is a photoreceptor specific promoter.

In additional embodiments, the compositions of the present disclosure include plasmids and vector comprising a nucleic acid molecule of the disclosure. In some embodiments, the vector is a viral vector such as a lentiviral vector or an adeno-associated vims (AAV) vector, (e.g., AAV2, AAV5, or AAV8 virus vector). In more embodiments, the methods include a nanoparticle or a dendrimer comprising the nucleic acid molecule of the disclosure.

CRX is an OTX-family homeodomain transcription factor required for appropriate development of retinal photoreceptor cells (Chen et al., Neuron, 19, 1017-1030; Furakawa et al., Cell, 91, 531-541). In vivo, CRX is specifically expressed in the retina and the pineal gland. Its function is primarily related to regulating gene expression in retinal photoreceptor cells necessary for proper vision and pinealocytes involved in circadian rhythms. Loss of CRX in mice results in loss of visual function (Furakawa et al., Nature genetics, 23, 466-470.) as the photoreceptors don't express necessary phototransduction genes and do not elaborate outer segments, specialized organelles containing visual pigments Opsins and phototransduction-related proteins.

In humans, in vivo the CRX gene (Gene ID: 1406; Ensembl: ENSG00000105392; MIM #602225, Dec. 31, 2019, incorporated herein by reference) is located on chromosome 19ql3.33 and encodes a 299 amino acid DNA binding protein. An exemplary amino acid sequence of human CRX is provided below, see UniProtKB No. 043186, as available on Dec. 31, 2019, incorporated herein by reference:

| | |
|---|---|
| SEQ | MMAYMNPGPHYSVNALALSGPSVDLMHQAVPYPSAPR |
| ID | KQRRERTTFTRSQLEELEALFAKTQYPDVYAREEVAL |
| NO: 1 | KINLPESRVQVWFKNRRAKCRQQRQQQKQQQQPPGGQ |
| | AKARPAKRKAGTSPRPSTDVCPDPLGISDSYSPPLPG |
| | PSGSPTTAVATVSIWSPASESPLPEAQRAGLVASGPS |
| | LTSAPYAMTYAPASAFCSSPSAYGSPSSYFSGLDPYL |
| | SPMVPQLGGPALSPLSGPSVGPSLAQSPTSLSGQSYG |
| | AYSPVDSLEFKDPTGTWKFTYNPMDPLDYKDQSAWKF |
| | QIL |

In some embodiments, the CRX protein comprises the amino acid sequence set forth as SEQ ID NO: 1. In other embodiments, the CRX protein comprises an amino acid sequence at least about 90%, 91%, 92%, 93%, 94%, 95%, 96%, 97%, 98% or 99% identical to SEQ ID NO: 1. In some non-limiting examples, the CRX protein is at least 95% identical to SEQ ID NO: 1. The CRX protein can function as a trans-activator.

CRX is a transcription factor that regulates the expression of a large number of rod and cone photoreceptor genes. Transactivation by CRX can be tested in vitro (using promoters that are CRX regulated to drive the expression of GFP or other reporter genes) or in vivo using models (including Crx-ko mice). This transcriptional activation function is needed for photoreceptor development and function.

SEQ ID NO: 1 is 299 amino acids in length. The DNA binding domain is at the N terminus and comprises residues 39 to 108, whereas transcriptional activation domain of the protein is located towards the C terminus (from residues 113 to 284). In some embodiments, a CRX protein of use is at least about 95%, 96%, 97%, 98%, or 99% identical to SEQ ID NO: 1, and includes residues 39 to 108 of SEQ ID NO: 1. In other embodiments, a CRX protein of use is at least about 95%, 96%, 97%, 98%, or 99% identical to SEQ ID NO: 1, and includes amino acids, and includes residues 113 to 284 of SEQ ID NO: 1. In further embodiments, a CRX protein of use is at least about 95% identical to SEQ ID NO: 1, and includes residues 39 to 108 and residues 113 to 284 of SEQ ID NO: 1. In more embodiments, a CRX protein of use is at least about 96%, 97%, 98% or 99% identical to SEQ ID NO: 1, and includes residues 39 to 108 and residues 113 to 284 of SEQ ID NO: 1. This CRX protein can function as a trans-activator.

In some embodiments, the CRX protein includes the OTX tail, residues 284 to 299 of SEQ ID NO: 1. In other embodiments, a CRX protein of use is at least about 95%, 96%, 97%, 98%, or 99% identical to SEQ ID NO: 1, and includes residues 284 to 299 of SEQ ID NO: 1. In further embodiments, a CRX protein of use is at least about 95% identical to SEQ ID NO: 1, and includes residues 39 to 108, residues 113 to 284 of SEQ ID NO: 1 and residues 284 to 299 of SEQ ID NO: 1. In more embodiments, a CRX protein of use is at least about 96%, 97%, 98% or 99% identical to SEQ ID NO: 2, and includes residues 39 to 108, residues 113 to 284, and residues 284 to 299 of SEQ ID NO: 2. This CRX protein can function as a trans-activator.

CRX protein exhibits high sequence homology in primates (chimpanzee 99%, crab-eating macaque 100%, gorilla 100%, marmoset 98%) and in model organisms (cat 93%, chicken 57%, dog 97%, mouse 89%, rat 97%, zebrafish 57%). Thus, in some embodiments, the CRX protein can include the corresponding amino acid from the CRX protein of another species. This CRX protein can function as a trans-activator.

Polynucleotides encoding a CRX protein are of use in the disclosed methods. These polynucleotides include DNA, cDNA, and RNA sequences that encode the CRX protein. Silent mutations in the coding sequence result from the degeneracy (codon redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (e.g., L. Stryer, 1988, Biochemistry, 3.sup.rd Edition, W. H. 5 Freeman and Co., NY). Degenerate variants are also of use in the methods disclosed herein.

Nucleic acid molecules encoding a CRX protein can readily be produced by one of skill in the art using the amino acid sequences provided herein and the genetic code. Nucleic acid sequences encoding the CRX protein can be prepared by any suitable method including, for example, cloning of appropriate sequences or by direct chemical synthesis by methods such as the phosphotriester method of Narang et al., Meth. Enzymol. 68:90-99, 1979; the phosphodiester method of Brown et al., Meth. Enzymol. 68:109-151, 1979; the diethylphosphoramidite method of Beaucage et al., Tetra. Lett. 22:1859-1862, 1981; the solid phase phosphoramidite triester method described by Beaucage & Caruthers, Tetra. Letts. 22 (20): 1859-1862, 1981, for example, using an automated synthesizer as described in, for example, Needham-VanDevanter et al., Nucl. Acids Res. 12:6159-6168, 1984 and the solid support method of U.S. Pat. No. 4,458,066. Chemical synthesis produces a single-strand (ss) oligonucleotide, which can be converted into double-strand (ds) DNA by hybridization with a complementary sequence or by polymerization with a DNA polymerase using the single strand as a template. Exemplary nucleic acids that include sequences encoding a CRX protein can be prepared by cloning techniques.

A nucleic acid molecule encoding a CRX protein can be cloned or amplified by in vitro methods, such as the polymerase chain reaction (PCR), the ligase chain reaction (LCR), the transcription-based amplification system (TAS), the self-sustained sequence replication system (3SR), and the replicase amplification system (QB). For example, a polynucleotide encoding the protein can be isolated by a polymerase chain reaction of cDNA using primers based on the DNA sequence of the molecule. A wide variety of cloning and in vitro amplification methodologies are well-known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, NY, 1989). Polynucleotides also can be isolated by screening genomic or cDNA libraries with probes selected from the sequences of the desired polynucleotide under stringent hybridization conditions.

Typically, a polynucleotide sequence encoding a CRX protein is operably linked to transcriptional control sequences including, for example a promoter and optionally a Woodchuck Hepatitis Virus (WHP) Posttranscriptional Regulatory Element (WPRE) or a polyadenylation signal.

In addition, a nucleic acid molecule of the disclosure may include a nucleic acid sequence encoding a short hairpin RNA (shRNA) specific for a mutant CRX. As another example, a nucleic acid sequence of the disclosure encodes a short interfering RNA (siRNA) specific for a mutant CRX. As another example, RNA (e.g., long noncoding RNA (IncRNA)) can be targeting mutant CRX with antisense oligonucleotides (ASOs) can be used. Processes for making ASOs targeted to RNAs are well known; see e.g. Zhou et al. 2016 Methods Mol Biol. 1402:199-213. Except as otherwise noted herein, therefore, the process of the present disclosure can be carried out in accordance with such processes. Methods of down-regulation or silencing genes are known in the art. For example, expressed protein activity can be down-regulated or eliminated using antisense oligonucleotides (ASOs), nucleotide aptamers, and RNA interference (RNAi) (e.g., small interfering RNAs (siRNA), short hairpin RNA (shRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14 (12): 807-15, describing targeting deoxyribonucleotide sequences; Lee et al. (2006) Curr Opin Chem Biol. 10, 1-8, describing aptamers; Reynolds et al. (2004) Nature Biotechnology 22 (3), 326-330, describing RNAi; Pushparaj and Melendez (2006) Clinical and Experimental Pharmacology and Physiology 33 (5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). Several siRNA molecule design programs using a variety of algorithms are known to the art (see e.g., Cenix algorithm, Ambion; BLOCK-IT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs. In each of the above embodiments, the wild-type CRX provided by the nucleic acid molecule of the disclosure is resistant to the shRNA or siRNA. Thus, only the expression levels of the mutant version of the CRX protein is reduced while the wild-type CRX protein expression is unaltered. In one example, the shRNA or siRNA targets the 3′ untranslated region (UTR) of mutant CRX. In another example, the nucleic acid sequence encoding the shRNA is SEQ ID NO: 3. Thus in embodiments which comprise the CRX specific shRNA, the CRX nucleic acid sequence operably linked to a photoreceptor specific promoter may be engineered such that the mRNA is resistant to the shRNA. Methods of designing interfering RNA resistant molecules are known in the art. In an exemplary embodiment, the promoter operably linked to the nucleic acid encoding a CRX specific shRNA is a U6 promoter.

Woodchuck Hepatitis Virus (WHV) Posttranscriptional Regulatory Element (WPRE) is a DNA sequence that, when transcribed, creates a tertiary structure enhancing expression. The sequence is commonly used in molecular biology to increase expression of genes delivered by viral vectors. WPRE is a tripartite regulatory element with gamma, alpha, and beta components. This sequence has 100% homology with base pairs 1093 to 1684 of the Woodchuck hepatitis B virus (WHV8) genome. A polyadenylation signal is a polynucleotide sequence that directs the addition of a series of nucleotides on the end of the mRNA transcript for proper processing and trafficking of the transcript out of the nucleus into the cytoplasm for translation.

Exemplary promoters include viral promoters, such as cytomegalovirus immediate early gene promoter (“CMV”), herpes simplex virus thymidine kinase (“tk”), SV40 early transcription unit, polyoma, retroviruses, papilloma vims, hepatitis B vims, and human and si mian immunodeficiency viruses. Other promoters include promoters isolated from mammalian genes, such as the immunoglobulin heavy chain, immunoglobulin light chain, T cell receptor, HLA DQ α and DQ β, β-interferon, interleukin-2, interleukin-2 receptor, MHC class II, HLA-DRa, β\hat{ }eúη, muscle creatine kinase, prealbumin (transthyretin), elastase I, metallothionein, collagenase, albumin, fetoprotein, β\hat{ }1oMη, c-fos, c-HA-ras, neural cell adhesion molecule (NCAM), alantitrypsin, H2B (TH2B) histone, type I collagen, glucose-regulated proteins (GRP94 and GRP78), rat growth hormone, human semm amyloid A (SAA), troponin I (TNI), platelet-derived growth factor, and dystrophin, as well as photoreceptor specific promoters.

The promoter can be either inducible or constitutive. An inducible promoter is a promoter that is inactive or exhibits low activity except in the presence of an inducer substance. Additional examples of promoters include, but are not limited to, MT II, MMTV, collagenase, stromelysin, SV40, murine MX gene, a-2-macroglobulin, MHC class I gene h-2kb, HSP70, proliferin, tetracycline inducible, tumor necrosis factor, or thyroid stimulating hormone gene promoter.

In some embodiments, the promoter is a photoreceptor specific promoter. In some embodiments, the photoreceptor specific promoter is not a promoter that CRX mutant or wild-type protein directly transactivates. Thus, in some embodiments, the promoter is not a CRX promoter. Thus, a photoreceptor specific promoter for use according to the disclosure includes, but is not limited to, GRK and GnbQ501/3.

In some embodiments the GnbQ501/3 promoter is at least about 95%, 96%, 97%, 98% or 99% identical to SEQ ID NO: 2. In other embodiments, the GnbQ501/3 promoter comprises a nucleic acid sequence at least about 90%, 91%, 92%, 93%, 94%, 95%, 96%, 97%, 98% or 99% identical to SEQ ID NO: 2. In some non-limiting examples, the GnbQ501/3 promoter is at least 95% identical to SEQ ID NO: 2.

In a preferred embodiment, the U6 promoter drives expression of the shRNA or siRNA targeting mutant CRX. In some embodiments the U6 promoter is at least about 95%, 96%, 97%, 98% or 99% identical to SEQ ID NO: 4. In other embodiments, the U6 promoter comprises a nucleic acid sequence at least about 90%, 91%, 92%, 93%, 94%, 95%, 96%, 97%, 98% or 99% identical to SEQ ID NO: 4. In some non-limiting examples, the U6 promoter is at least 95% identical to SEQ ID NO: 4.

Optionally, transcription control sequences include one or more enhancer elements, which are binding recognition sites for one or more transcription factors that increase transcription above that observed for the minimal promoter alone, and also be operably linked to the polynucleotide encoding the photoreceptor specific promoter and/or the nucleic acid molecule encoding the CRX protein. With regard to the nucleic acid molecule encoding the CRX protein, introns can also be included that help stabilize mRNA and increase expression.

The term “tetracycline-controlled transactivator” (tTa) refers to a fusion protein used to control nucleic acid expression in the presence or absence of doxycycline, tetracycline and related compounds. The tTa includes a tet repressor (tetr) fused to any domain capable of activating transcription. The tTa may include a tetR fused to a c-terminal portion of VP16, including tTa1, tTa2, tTa3, tTa4, etc. (see, e.g., Baron, U. et al., Nuc. Acid Res., 25 (14): 2723-2729, 1997).

Thus, activation of a first promoter of the nucleic acid as disclosed herein results in expression of the tetracycline-controlled transactivator which then is available to bind the tTA-responsive (TRE) promoter and regulate expression of CRX. The tet response element-based system is was first described by Gossen and Bujuard (Proc. Natl. Acad. Sci. USA, 89:5547-5551, 1992). The tet system includes several advantages. The elements required for control of the tet system are derived from a prokaryotic organism, and thus, there is no endogenous expression of these control elements in mammalian cells. In addition, a preferred effector doxycycline (dox), a tet derivative, is an FDA approved drug that can regulate the transgene expression at very low concentrations without producing detectable side effects (Corti et al., Nat. Biotechnol., 17: 349-354, 1999; Hasan et al., Genesis, 29: 116-122, 2001). Activation of the tet-regulated system by dox is dose-dependent and gene expression can be controlled over a narrow window of dox concentrations (Urlinger et al., Proc. Natl. Acad. Sci. USA, 97: 7963-7969, 2000). The genes required for the tet system are small compared to elements required for other systems, which is advantageous given the limited insert size of approximately 4.5 kb, for rAAV vectors (Baron et al., Methods Enzymol., 327: 401-421, 2000).

The tet inducible system includes two components, the tet-controlled transactivator protein (tTA), and the tet-regulated element (TRE). The tTA is a fusion protein of the tet repressor DNA binding domain of Escherichia coli (TetR) and the C-terminal transcriptional activator domain of the VP16 protein from herpes simplex virus. The TRE region includes seven copies of the tetracycline resistance operator binding sites and a minimal promoter region (e.g., a minimal cytomegalovirus (CMV) promoter) that contains the TATA box and transcription start sites. In the absence of tet or dox, tTA can bind a tetracycline operator (tetO) sequence located in front of the minimal promoter and stimulate transcription of the transgene. Dox prevents this binding and consequently abolishes transcription because the minimal promoter by itself is inactive. This tet-off system has also been modified to make a tet-on system. When the tTA is replaced by a mutated transactivator, rtTA, the promoter regulation by the transactivator is reversed so that transgene expression occurs in the presence of dox and is shut off in the absence of dox. (Gossen et al., Proc. Natl. Acad. Sci. USA, 89:5547-5551, 1992; Urlinger et al., Proc. Natl. Acad. Sci. USA, 97: 7963-7969, 2000; Baron et al., Methods Enzymol., 327: 401-421, 2000).

The tet-off and tet-on systems have been studied in the context of various viral vectors. In some cases, the two components of the tet system have been cloned into separate viruses, which requires coinfection of the target cells by both viruses to obtain regulatable transgene expression (see, for example, Bohl et al., Hum. Gene Ther., 8:195-204, 1997). Another strategy is to combine both components into one self-regulating virus so that target cells only need to be infected by one virus to allow regulatable expression. (see, for example, Corti et al., Nat. Biotechnol., 17: 349-354, 1999).

The terms “tetracycline-off” and “tet-off” refer to a tetracycline inducible promoter system including tetracycline response elements that allow transcription of a nucleic acid that is operatively linked to the promoter system in the absence of doxycycline or tetracycline, and related compounds. In the presence of doxycycline or tetracycline transcription of the operatively linked nucleic acid is turned “off” in a non-leaky promoter system. The term “non-leaky” refers to control of expression of the operatively linked nucleic acid so that in the presence of doxycycline or tetracycline, expression of the nucleic acid is about 10%, preferably about 5%, or less in comparison to expression of the nucleic acid in the absence of doxycycline or tetracycline.

In some embodiments, the disclosed nucleic acid molecules can be incorporated into a vector capable of expression in a host cell, using established molecular biology procedures. For example, nucleic acids, such as cDNAs, that encode a CRX protein can be manipulated with standard procedures, such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate, or use of specific oligonucleotides in combination with PCR or other in vitro amplification. These vectors can include a photoreceptor specific promoter operably linked to a nucleic acid molecule encoding a CRX protein and other elements as discussed above.

Exemplary procedures sufficient to guide one of ordinary skill in the art through the production of a vector capable of expression in a host cell that includes a photoreceptor specific promoter, and/or a polynucleotide sequence of the disclosure can be found, for example, in Sambrook et al., Molecular Cloning: A Laboratory Manual, 2d ed., Cold Spring Harbor Laboratory Press, 1989;

Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2003); and Ausubel et al., Short Protocols in Molecular Biology: A Compendium of Methods from Current Protocols in Molecular Biology, 4th ed., Wiley & Sons, 1999.

It may be desirable to include a polyadenylation signal to effect proper termination and polyadenylation of the gene transcript. Exemplary polyadenylation signals have been isolated from beta globin, bovine growth hormone, SV40, and the herpes simplex vims thymidine kinase genes.

The disclosed nucleic acid molecules can be included in a nanodispersion system, see, e.g., U.S. Pat. No. 6,780,324; U.S. Pat. Publication No. 2009/0175953. For example, a nanodispersion system includes a biologically active agent and a dispersing agent (such as a polymer, copolymer, or low molecular weight surfactant). Exemplary polymers or copolymers include polyvinylpyrrolidone (PVP), poly(D,L-lactic acid) (PLA), poly(D,L-lactic-co-glycolic acid (PLGA), poly(ethylene glycol). Exemplary low molecular weight surfactants include sodium dodecyl sulfate, hexadecyl pyridinium chloride, polysorbates, sorbitans, poly(oxyethylene) alkyl ethers, poly(oxyethylene) alkyl esters, and combinations thereof. In one example, the nanodispersion system includes PVP and ODP or a variant thereof (such as 80/20 w/w). In some examples, the nanodispersion is prepared using the solvent evaporation method, see for example, Kanaze et al., Drug Dev. Indus. Pharm. 36:292-301, 2010; Kanaze et al., J. Appl. Polymer Sci. 102:460-471, 2006

Accordingly, the present disclosure relates to a vector comprising a nucleic acid sequence as described herein. The nucleic acid sequences of the present disclosure may be generated by cloning a disclosed nucleic acid sequence into a viral vector. The viral vector of the disclosure may be a recombinant adeno-associated (rAAV or AAV) vector. AAV vectors are small, single-stranded DNA viruses which require helper virus to facilitate efficient replication. The viral vector comprises a vector genome and a protein capsid. The viral vector capsid may be supplied from any of the AAV serotypes known in the art, including presently identified human and non-human AAV serotypes and AAV serotypes yet to be identified. Virus capsids may be mixed and matched with other vector components to form a hybrid viral vector, for example the ITRs and capsid of the viral vector may come from different AAV serotypes. In one aspect, the ITRs can be from an AAV2 serotype while the capsid is from, for example, an AAV2 or AAV5 serotype. In addition, one of skill in the art would recognize that the vector capsid may also be a mosaic capsid (e.g.: a capsid composed of a mixture of capsid proteins from different serotypes), or even a chimeric capsid (e.g.: a capsid protein containing a foreign or unrelated protein sequence for generating markers and/or altering tissue tropism). It is contemplated that the viral vector of the disclosure may comprise an AAV2 capsid. It is further contemplated that the invention may comprise an AAV5 capsid.

According to the invention the term “AAV” refers to AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), and AAV type 8 (AAV-8) and AAV type 9 (AAV9). The genomic sequences of various serotypes of AAV, as well as the sequences of the native terminal repeats (TRs), Rep proteins, and capsid subunits are known in the art. Such sequences may be found in the literature or in public databases such as GenBank. See, e.g., GenBank Accession Numbers NC_001401 (AAV-2), AF043303 (AAV2), and NC_006152 (AAV-5).

As used herein, an “AAV vector” refers to an AAV vector comprising the polynucleotide of interest (i.e. a heterologous polynucleotide) for the genetic expression in a cone and/or rod cell. The rAAV vectors contain 5′ and 3′ adeno-associated virus inverted terminal repeats (ITRs), and the polynucleotide of interest (CRX) operatively linked to sequences, which regulate its expression in a target cell.

In a specific aspect, the AAV vector of the invention is an AAV2 serotype.

In another aspect, the AAV vector of the invention is an AAV2/5 or AAV2/8 serotype. The AAV2/5 or AAV2/8 vectors of the invention are produced using methods known in the art. In short, the methods generally involve (a) the introduction of the AAV vector into a host cell, (b) the introduction of an AAV helper construct into the host cell, wherein the helper construct comprises the viral functions missing from the AAV vector and (c) introducing a helper virus into the host cell. All functions for AAV virion replication and packaging need to be present, to achieve replication and packaging of the AAV vector into AAV virions. The introduction into the host cell can be carried out using standard virological techniques simultaneously or sequentially. Finally, the host cells are cultured to produce AAV virions and are purified using standard techniques such as cesium chloride (CsCl) gradients. Residual helper virus activity can be inactivated using known methods, such as for example heat inactivation. The purified AAV virion is then ready for use in the methods of the invention.

The vector may also comprise regulatory sequences allowing expression and, secretion of the encoded protein, such as e.g., a promoter, enhancer, polyadenylation signal, internal ribosome entry sites (IRES), sequences encoding protein transduction domains (PTD), and the like. In this regard, the vector comprises a promoter region, operably linked to the polynucleotide of interest, to cause or improve expression of the protein in infected cells. Such a promoter may be ubiquitous, tissue-specific, strong, weak, regulated, chimeric, inducible, etc., to allow efficient and suitable production of the protein in the infected tissue. The promoter may be homologous to the encoded protein, or heterologous, including cellular, viral, fungal, plant or synthetic promoters. The preferred promoter for use in the present invention is chosen among promoter able to drive expression in cone and rod cells such as retinitis pigmentosa 1 (RP1) or the human Rhodopsin kinase 1 (GRK1). In a preferred aspect the invention deals with an AAV vector wherein the polynucleotide encoding CRX is under the control of the human Rhodopsin kinase 1 (GRK1) promoter.

Thus, genetic modification of a photoreceptor cell can be accomplished by transducing a cell with a recombinant DNA construct. In certain embodiments, a retroviral vector (either gamma-retroviral or lentiviral) is employed for the introduction of the DNA construct into the cell. For example, a polynucleotide comprising the regulatory sequences of the disclosure and/or a nucleic acid sequence encoding a therapeutic biologic can be cloned into a retroviral vector and expression can be driven from its endogenous promoter, from the retroviral long terminal repeat, or from a promoter specific for a target cell type of interest. Other viral vectors, or non-viral vectors may be used as well.

For initial genetic modification of a photoreceptor cell, a retroviral vector is generally employed for transduction, however any other suitable viral vector or non-viral delivery system can be used. The nucleic acid sequences can be constructed with an auxiliary molecule (e.g., a cytokine) in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In certain embodiments, any vector disclosed herein can comprise a P2A peptide. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Nat. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J Clin. Invest. 89:1817.

Other transducing viral vectors can be used to genetically modify a target cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a photoreceptor cell. For example, a nucleic acid molecule of the disclosure can be introduced into a target cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue, are injected systemically, or incorporated into an implantable tissue which is the administered to a subject. Recombinant receptors can also be derived or obtained using transposases or targeted nucleases (e.g. Zinc finger nucleases, meganucleases, or TALE nucleases, CRISPR). Transient expression may be obtained by RNA electroporation.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to forma single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

Any targeted genome editing methods can be used to place presently disclosed nucleic acid sequence at one or more endogenous gene loci of a presently disclosed target cell. In certain embodiments, a CRISPR system is used to deliver presently disclosed engineered gene circuit to one or more endogenous gene loci of a presently disclosed target cell. In certain embodiments, zinc-finger nucleases are used to deliver presently disclosed nucleic acid sequences to one or more endogenous gene loci of a presently disclosed target cell. In certain embodiments, a TALEN system is used to deliver presently disclosed engineered gene circuit to one or more endogenous gene loci of a presently disclosed target cell.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

The present disclosure also provides pharmaceutical compositions. The pharmaceutical composition comprises a vector or viral particle comprising a nucleic acid sequence according to the present disclosure, as an active component, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

Compositions comprising vector comprising a nucleic acid sequence according to the present disclosure can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The invention also deals with compositions and pharmaceutical compositions comprising the viral vectors of the invention. The pharmaceutical composition are formulated together with a pharmaceutically acceptable carrier. The compositions can additionally contain one or more other therapeutic agents that are suitable for treating or preventing, for example, CRX-associated IRD. Pharmaceutically acceptable carriers enhance or stabilize the composition, or can be used to facilitate preparation of the composition. Pharmaceutically acceptable excipient refers to a non-toxic solid, semi-solid or liquid filler, diluent, encapsulating material or formulation auxiliary of any type. Pharmaceutically acceptable excipients that may be used in the compositions of the invention include, but are not limited to solvents, surfactants, dispersion media, coatings, antibacterial and antifungal agents, isotonic and absorption delaying agents, and the like that are physiologically compatible ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, di sodium hydrogen phosphate, potassium hydrogen phosphate, sodium chloride, zinc salts, silica, colloidal silica, magnesium trisilicate, polyvinyl pyrrolidone, cellulose-based substances (e.g.: sodium carboxymethylcellulose), polyethylene glycol, polyacrylates, waxes, polyethylene-polyoxypropylene-block polymers, polyethylene glycol and wool fat. The pharmaceutical composition according to the present invention may further comprise antioxidant agents, including, but not limited to, ascorbic acid, ascorbyl palmitate, butylated hydroxytoluene, potassium sorb ate or Rosmarinus officinalis extracts.

The pharmaceutical composition according to the present disclosure may further comprise pharmaceutically acceptable salts, including, but not limited to, acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The excipient can also be a solvent or dispersion medium containing, for example, water, ethanol, polyol (e.g.: glycerol, propylene glycol, and liquid polyethylene glycol, and the like), suitable mixtures thereof, and vegetables oils such as oleic acid. The proper fluidity can be maintained, for example, by the use of a coating, such as lecithin (i.e., soy lecithin or de-greased soy lecithin), by the maintenance of the required particle size in the case of dispersion and by the use of surfactants.

The vector or the pharmaceutical composition of the present disclosure can be administered by a variety of methods known in the art. The route and/or mode of administration vary depending upon the desired results. It is preferred that administration be subretinal. The pharmaceutically acceptable carrier should be suitable for subretinal, intravitreal, intravenous, sub-cutaneous or topical administration.

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition.

Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. For delayed release, the vector may be included in a pharmaceutical composition, which is formulated for slow release, such as in microcapsules formed from biocompatible polymers or in liposomal carrier systems according to methods known in the art.

Pharmaceutical compositions of the disclosure can be prepared in accordance with methods well known and routinely practiced in the art.

Pharmaceutical compositions are preferably manufactured under GMP conditions. Typically, a therapeutically effective dose or efficacious dose of the viral vector is employed in the pharmaceutical compositions of the invention. The viral vectors may be formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

As used herein, “therapeutically effective amount” means an amount of a vector (e.g. AAV) comprising a nucleic acid sequence as disclosed herein that provides a therapeutic benefit to the subject. In one embodiment, the term “effective amount” means level or amount of vector that is aimed at, without causing significant negative or adverse side effects to the target, (1) delaying or preventing the onset of CRX-associated retina disease; (2) slowing down or stopping the progression, aggravation, or deterioration of one or more symptoms of CRX-associated retina disease (3) bringing about ameliorations of the symptoms of CRX-associated retina disease; (4) reducing the severity or incidence of CRX-associated retina disease; or (5) curing the CRX-associated retina disease.

The doses of vectors may be adapted depending on the disease condition, the subject (for example, according to his weight, metabolism, etc.), the treatment schedule, etc. A preferred effective dose within the context of this invention is a dose allowing an optimal expression in photoreceptor cells.

Treatment dosages need to be titrated to optimize safety and efficacy. For subretinal administration with a viral vector, the dosage may range from 1×108 vector genomes (vg)/eye to 1×1012 vg/eye. For example the dosage may be, 1×108 vg/eye, 1.5×108 vg/eye, 2×108 vg/eye, 2.5×108 vg/eye, 3×108 vg/eye, 4×108 vg/eye, 5×108 vg/eye, 6×108 vg/eye, 7.5×108 vg/eye, 8×108 vg/eye, 9×108 vg/eye, 0.5×109 vg/eye, 1×109 vg/eye, 1.5×109 vg/eye, 2×109 vg/eye, 2.5×109 vg/eye, 3×109 vg/eye, 4×109 vg/eye, 5×109 vg/eye, 6×109 vg/eye, 7.5×109 vg/eye, 8×109 vg/eye, 9×109 vg/eye, 0.5×1010 vg/eye, 1.5×1010 vg/eye, 2×1010 vg/eye, 2.5×1010 vg/eye, 3×1010 vg/eye, 4×1010 vg/eye, 5×1010 vg/eye, 6×1010 vg/eye, 7.5×1010 vg/eye, 8×1010 vg/eye, 9×1010 vg/eye, 0.5×1011 vg/eye, 1.5×1011 vg/eye, 2×1011 vg/eye, 2.5×1011 vg/eye, 3×1011 vg/eye, 4×1011 vg/eye, 5×1011 vg/eye, 6×1011 vg/eye, 7.5×1011 vg/eye, 8×1011 vg/eye, 9×1011 vg/eye, 0.5×1012 vg/eye, 1×1012 vg/eye.

According to a preferred embodiment of the invention, the vectors or the composition comprising said vector is administrated in a quantity between 1×109 and 1×1012 vg/eye.

Administering the vector of the invention vector to the subject may be done by direct subretinal injection for an expression of CRX in cone and rod cells.

According to another aspect, the vector or the composition for use according to the invention is administered before disease onset and as long as there is a need to prevent this onset.

According to another aspect, the vector or the composition for use according to the invention is administered after initiation of photoreceptor degeneration more particularly after initiation of photoreceptor degeneration as long as there are functional cone and/or photoreceptors and as long as there is a need.

The viral vectors described herein are mainly used as one-time doses per eye, with the possibility of repeat dosing to treat regions of the retina that are not covered in the previous dosing. The dosage of administration may vary depending on whether the treatment is prophylactic or therapeutic.

In some embodiments, if the nucleic acid molecule is included in an AAV vector, an effective amount to achieve a change will be about 1×108 vector genomes or more, in some cases about 1×109, about 1×1010, about 1×10 11, about 1×1012, or about 1×1013 vector genomes or more, in certain instances, about 1×1014 vector genomes or more, and usually no more than about 1×10 15 vector genomes. In some embodiments, the amount of vector that is delivered is about 1×10+vectors or less, for example about 1×1013, about 1×1012, about 1×1011, about 1× 1010, or about 1×109 vectors or less, in certain instances about 1×108 vectors, and typically no less than 1×108 vectors. In some non-limiting examples, the amount of vector genomes that is delivered is about 1×1010 to about 1×1011 vectors. In additional non-limiting examples, the amount of vector that is delivered is about 1×1010 to about 1×1012 vector genomes.

In some embodiments, the amount of pharmaceutical composition to be administered may be measured using multiplicity of infection (MOI). In some embodiments, MOI refers to the ratio, or multiple of vector or viral genomes to the cells to which the nucleic may be delivered. In some embodiments, the MOI may be about 1×10 6. In some cases, the MOI can be about 1×10 5 to about 1×10 7. In some cases, the MOI may be about 1×10 4 to about 1×10 8. In some cases, recombinant viruses of the disclosure are at least about 1×10 1, about 1×10 2, about 1×10 3, about 1×10 4, about 1×10 5, about 1×106, about 1×10 7, about 1×10 8, about 1×10 9, about 1×10 10, about 1×10 11, about 1×10 12, about 1×10 13, about 1×10 14, about 1×10 15, about 1×10 16, about 1×10 17, and about 1×10 18 MOI. In some cases, recombinant viruses of this disclosure are about 1×10 8 to 1×10 14 MOI.

Briefly, a subject for intravitreal injection may be prepared for the procedure by pupillary dilation, sterilization of the eye, and administration of anesthetic. Any suitable mydriatic agent known in the art may be used for pupillary dilation. Adequate pupillary dilation may be confirmed before treatment. Sterilization may be achieved by applying a sterilizing eye treatment, e.g., an iodide-containing solution such as povidone-iodine (BETADINE®). A similar solution may also be used to clean the eyelid, eyelashes, and any other nearby tissues (e.g., skin). Any suitable anesthetic may be used, such as lidocaine or proparacaine, at any suitable concentration.

Anesthetic may be ad ministered by any method known in the art, including without limitation topical drops, gels or jellies, and subconjunctival application of anesthetic.

Prior to injection, a sterilized eyelid speculum may be used to clear the eyelashes from the area. The site of the injection may be marked with a syringe. The site of the injection may be chosen based on the lens of the patient. For example, the injection site may be 3-3.5 mm from the limus in pseudophakic or aphakic patients, and 3.5-4 mm from the limbus in phakic patients. The patient may look in a direction opposite the injection site. During injection, the needle can be inserted perpendicular to the sclera and pointed to the center of the eye. The needle can be inserted such that the tip ends in the vitreous, rather than the subretinal space. Any suitable volume known in the art for injection may be used. After injection, the eye can be treated with a sterilizing agent such as an antibiotic. The eye can also be rinsed to remove excess sterilizing agent.

The subject can be administered additional therapeutic agents. Additional agents that can be ad ministered to the subject include antibacterial and antifungal antibiotics, as well as non-steroidal anti-inflammatory agents to reduce risk of infection and inflammation. Additional agents can be ad ministered by any route. The additional agents can be formulated separately, or in the same composition.

Agents of use include antibiotics such as minoglycosides (for example, amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (for example, azidamfenicol, chloramphenicol, florfenicol, thiamphenicol), ansamycins (for example, rifamide, rifampin, rifamycin sv, rifapentine, rifaximin), β-lac tarns (for example, carbacephems (e.g., loracarbef), carbapenems (for example, biapenem, imipenem, meropenem, panipenem), cephalosporins (for example, cefaclor, cefadroxil, cefamandole, cefatrizine, cefazedone, cefazolin, cefcapene pivoxil, cefclidin, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (for example, cefbuperazone, cefmetazole, cefininox, cefotetan, cefoxitin), monobactams (for example, aztreonam, carumonam, tigemonam), oxacephems, flomoxef, moxalactam), penicillins (for example, amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin g benzathine, penicillin g benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), other (for example, ritipenem), lincosamides (for example, clindamycin, lincomycin), macrolides (for example, azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (for example, amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (for example, apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin). Agents of use also include synthetic antibacterials, such as 2,4-Diaminopyrimidines (for example, brodimoprim, tetroxoprim, trimethoprim), nitrofurans (for example, furaltadone, furazolium chloride, nifuradene, nifuratel, nifurfoline, nifurpirinol, nifurprazine, nifurtoinol, nitrofurantoin), quinolones and analogs (for example, cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (for example, acetyl sulfamethoxypyrazine, benzylsulfamide, chloramine-b, chloramine-t, dichloramine t, mafenide, 4′(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidocchrysoidine, sulfamoxole, sulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (for example, acedapsone, acediasulfone, acetosulfone sodium, dapsone, diathymosulfone, glucosulfone sodium, solasulfone, succisulfone, sulfanilic acid, psulfanilylbenzylamine, sulfoxone sodium, thiazolsulfone), and others (for example, clofoctol, hexedine, methenamine, methenamine anhydromethylene-citrate, methenamine hippurate, methenamine mandelate, methenamine sulfosalicylate, nitroxoline, taurolidine, xibornol).

Additional agents of use include antifungal antibiotics such as polyenes (for example, amphotericin B, candicidin, dennostatin, filipin, fungichromin, hachimycin, hamycin, lucensomycin, mepartricin, natamycin, nystatin, pecilocin, perimycin), others (for example, azaserine, griseofulvin, oligomycins, neomycin undecylenate, pyrrolnitrin, siccanin, tubercidin, viridin) allylamines (for example, butenafine, naftifine, terbinafine), imidazoles (for example, bifonazole, butoconazole, chlordantoin, chlormiidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (for example, tolciclate, tolindate, tolnaftate), triazoles (for example, fluconazole, itraconazole, saperconazole, terconazole) others (for example, acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

Antineoplastic agents can also be of use including (1) antibiotics and analogs (for example, aclacinomycins, actinomycin, anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycines, peplomycin, pirarubicin, plicamycin, porfiromycin, puromycin, streptonigrin, streptozocin, tubercidin, zinostatin, zorubicin), (2) anti metabolites such as folic acid analogs (for example, denopterin, edatrexate, methotrexate, piritrexim, pteropterin, trimetrexate), (3) purine analogs (for example, cladribine, fludarabine, 6-mercaptopurine, thiamiprine, thioguanine), (4) pyrimidine analogs (for example, ancitabine, azacitidine, 6-azauridine, carmofur, cytarabine, doxifluridine, emitefur, enocitabine, floxuridine, fluorouracil, gemcitabine, tagafur).

Steroidal anti-inflammatory agents can also be used such as 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, cyclosporine, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

In addition, non-steroidal anti-inflammatory agents can be used. These include aminoarylcarboxylic acid derivatives (for example, enfenamic acid, etofenamate, flufenamic acid, isonixin, meclofenamic acid, mefenamic acid, niflumic acid, talniflumate, terofenamate, tolfenamic acid), arylacetic acid derivatives (for example, aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (for example, bumadizon, butibufen, fenbufen, xenbucin), arylcarboxylic acids (for example, clidanac, ketorolac, tinoridine), arylpropionic acid derivatives (for example, alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (for example, difenamizole, epirizole), pyrazolones (for example, apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (for example, acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (for example, ampiroxicam, droxicam, isoxicam, lornoxicam, piroxicam, tenoxicam), .epsilon.acetamidocaproic acid, s-adenosylmethionine, 3-amino-4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

### II. Methods

The disclosure provides methods for treating a disease associated with a genetic mutation, substitution, or deletion of the CRX gene that affects retinal photoreceptor cells, wherein the methods comprise administering to a subject in need of such treatment a vector that includes a nucleic acid sequence as disclosed herein, thereby treating the subject. The CRX gene provides instructions for making a protein called the cone-rod homeobox protein. This protein is found in the eyes, specifically in the light-sensitive tissue at the back of the eye called the retina. The cone-rod homeobox protein attaches (binds) to specific regions of DNA and helps control the activity of particular genes. On the basis of this action, this protein is called a transcription factor.

In the retina, the cone-rod homeobox protein is necessary for the normal development of light-detecting cells called photoreceptors. Through its actions as a transcription factor, the cone-rod homeobox protein helps photoreceptor cells mature into two types: rods and cones. Rods are needed for vision in low light, while cones are needed for vision in bright light, including color vision. The protein also helps maintain these cells and preserve vision.

More than 20 mutations in the CRX gene have been found to cause cone-rod dystrophy. The problems associated with this condition include a loss of visual sharpness (acuity), an increased sensitivity to light (photophobia), and impaired color vision. These vision problems worsen over time. Cone-rod dystrophy is caused by mutations that occur in one of the two copies of the CRX gene in each cell. CRX gene mutations are responsible for about one-quarter of the cases of a form of the condition called autosomal dominant cone-rod dystrophy. These mutations lead to a reduction in the amount of functional cone-rod homeobox protein that is available to regulate other genes in the retina. As a result, maintenance of the rod and cone cells is insufficient and these cells deteriorate over time, leading to the vision problems characteristic of cone-rod dystrophy. It is believed that there is enough cone-rod homeobox protein function to allow for photoreceptor cell differentiation, but long-term maintenance of the cells cannot be sustained.

Several CRX gene mutations have been found to cause different forms of vision loss in different individuals. It is unclear how mutations in the CRX gene can cause different eye disorders.

In addition to cone-rod dystrophy, other CRX-mediated retinopathies include but are not limited to leber congenital amaurosis, and retinitis pigmentosa. In a preferred embodiment, the disease is achromatopsia. Other diseases associated with a genetic mutation, substitution, or deletion that affects retinal cone cells include achromatopsia, Leber congenital amaurosis, cone-rod dystrophy, maculopathies, age-related macular degeneration and retinitis pigmentosa, including X-linked retinitis pigmentosa.

A “subject” to be treated by the methods of the invention can mean either a human or non-human animal. A “nonhuman animal” includes any vertebrate or invertebrate organism. In some embodiments, the nonhuman animal is an animal model of retinal disease. Various large animal models are available for the study of AAV-mediated gene-based therapies in the retina. Stieger K. et al. AAV-mediated gene therapy for retinal disorders inlarge animal models. ILAR J. 50 (2): 206-224 (2009). The promoters of the invention are described supra. “Treating” a disease (such as, for example, a CRX-mediated retinopathy) means alleviating, preventing, or delaying the occurrence of at least one sign or symptom of the disease. A “sign” of a disease is a manifestation of the disease that can be observed by others or measured by objective methods, such as, e.g., electroretinography or behavioral testing. A “symptom” of a disease is a characteristic of the disease that is subjectively perceived by the subject.

In some embodiments, administration of a vector comprising a nucleic acid sequence as disclosed herein to a subject having or suspected of having a CRX-meditated retinopathy increases the rod outer segment (OS) thickness of the retina relative to the to the OS thickness prior to administration of the vector. In some embodiments, administration of a vector comprising a nucleic acid sequence as disclosed herein to a subject having or suspected of having a CRX-meditated retinopathy increases the Rhodopsin expression in photoreceptor cells relative to Rhodopsin expression in photoreceptor cells prior to administration of the vector. In some embodiments, administration of a vector comprising a nucleic acid sequence as disclosed herein to a subject having or suspected of having a CRX-meditated retinopathy results in improved photoreceptor cell morphology relative to photoreceptor cell morphology prior to administration of the vector. In some embodiments, administration of a vector comprising a nucleic acid sequence as disclosed herein to a subject having or suspected of having a CRX-meditated increases rod and/or cone function as determined by an electroretinogram relative to the rod and/or cone function prior to administration of the vector. In some embodiments, administration of a vector comprising a nucleic acid sequence as disclosed herein to a subject having or suspected of having a CRX-meditated increases visual function relative to the visual function prior to administration of the vector. In some embodiments, administration of a vector comprising a nucleic acid sequence as disclosed herein to a subject having or suspected of having a CRX-meditated increases CRX target gene expression relative to the CRX mediated gene expression prior to administration of the vector.

In the methods of treatment of the present invention, administering of a vector can be accomplished by any means known in the art. In preferred embodiments, the administration is by subretinal injection. In certain embodiments, the subretinal injection is delivered preferentially to one or more regions where cone density is particularly high (such as e.g., the tapetal zone superior to the optic disc). In other embodiments, the administration is by intraocular injection, intravitreal injection, or intravenous injection. Administration of a vector to the retina may be unilateral or bilateral and may be accomplished with or without the use of general anesthesia.

In the methods of treatment of the present disclosure, the volume of vector delivered may be determined based on the characteristics of the subject receiving the treatment, such as the age of the subject and the volume of the area to which the vector is to be delivered. It is known that eye size and the volume of the subretinal space differ among individuals and may change with the age of the subject. In embodiments wherein the vector is administered subretinally, vector volumes may be chosen with the aim of covering all or a certain percentage of the subretinal space, or so that a particular number of vector genomes is delivered.

In the methods of treatment of the present invention, the concentration of vector that is administered may differ depending on production method and may be chosen or optimized based on concentrations determined to be therapeutically effective for the particular route of administration.

Methods described herein are generally performed on a subject in need thereof. A subject in need of the therapeutic methods described herein can be a subject having, diagnosed with, suspected of having, or at risk for developing a CRX-mediated retinopathy. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to vectors of the disclosure. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or from a randomized group of subjects. A reference value can be used in place of a control or reference sample, which was previously obtained from a healthy subject or a group of healthy subject. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### Equivalents

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The terms “patient,” “subject,” “individual,” and the like are used interchangeably herein, and refer to any animal or cells thereof whether in vitro or in situ, amenable to the methods described herein. In certain non-limiting embodiments, the patient, subject or individual is a human.

As used herein, the term “subject” refers to a mammal, preferably a human. The mammals include, but are not limited to, humans, primates, livestock, rodents, and companion animals. A subject may be waiting for medical care or treatment, may be under medical care or treatment, or may have received medical care or treatment.

The following definitions and methods are provided to better define the present disclosure and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host (target) cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 50-99% identity to the reference sequence described herein and screen such for desired phenotypes according to methods routine in the art.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Donor DNA”, “donor template” and “repair template” as used interchangeably herein refers to a double-stranded DNA fragment or molecule that includes at least a portion of the gene of interest. The donor DNA may encode a full-functional protein or a partially-functional protein.

“Endogenous gene” as used herein refers to a gene that originates from within an organism, tissue, or cell. An endogenous gene is native to a cell, which is in its normal genomic and chromatin context, and which is not heterologous to the cell. Such cellular genes include, e.g., animal genes, plant genes, bacterial genes, protozoal genes, fungal genes, mitochondrial genes, and chloroplastic genes.

“Functional” and “full-functional” as used herein describes protein that has biological activity. A “functional gene” refers to a gene transcribed to mRNA, which is translated to a functional protein.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed.

“Genome editing” as used herein refers to changing the endogenous DNA of a cell. Genome editing may include the addition of nucleic acids, deletion of nucleic acids, or restoring a mutant gene. Genome editing may include knocking out a gene, such as a mutant gene or a normal gene, or knocking-in a heterologous gene or protein encoding region thereof.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery may repair the break by homologous recombination, which is enhanced several orders of magnitude in the presence of DNA cleavage. When the homologous DNA piece is absent, non-homologous end joining may take place instead.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

“Nuclease mediated NHEJ” as used herein refers to NHEJ that is initiated after a nuclease, such as a Cas9, cuts double stranded DNA.

“Site-specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas-based systems.

“Target region” as used herein refers to the region of the target gene to which the site-specific nuclease is designed to bind and cleave.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by lle, Leu by lle, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65 □C in a 6 X SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65 □C in the salt conditions of a 6×SSC, then the two sequences will not hybridize. On the other hand, if the melting temperature is above 65 □C in the same salt conditions, then the sequences will hybridize. In general, the melting temperature for any hybridized DNA:DNA sequence can be determined using the following formula: Tm=81.5 □C+16.6 (log10[Na+])+0.41 (fraction G/C content)−0.63(% formamide)−(600/l). Furthermore, the Tm of a DNA: DNA hybrid is decreased by 1-1.5 □C for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

A “promoter”, as used herein refers to a sequence that regulates transcription of an operably-linked gene, or nucleotide sequence encoding a protein, etc. Promoters provide the sequence sufficient to direct transcription, as well as, the recognition sites for RNA polymerase and other transcription factors required for efficient transcription and can direct cell specific expression. In addition to the sequence sufficient to direct transcription, a promoter sequence of the disclosure can also include sequences of other regulatory elements that are involved in modulating transcription (e.g.: enhancers, kozak sequences and introns). In addition, standard techniques are known in the art for creating functional promoters by mixing and matching known regulatory elements.

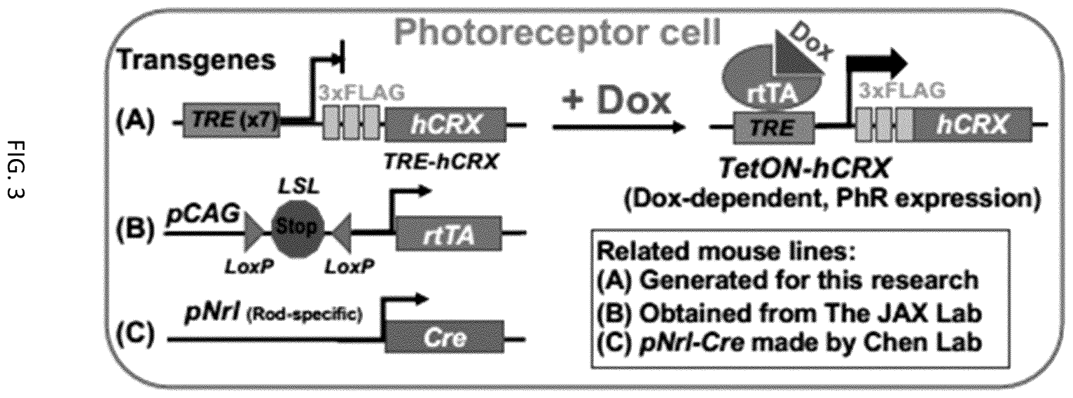

“Photoreceptor cell specific,” as used herein, means that the promoter preferentially initiates transcription in photoreceptor cells as opposed to other cell types. In an exemplary embodiment, the promoter has detectable activity only in photoreceptor cells. The retina contains three kinds of photoreceptors: rod cells, cone cells, and photoreceptive ganglion cells. Cone cells are of three types: S-cone cells, M-cone cells, and L-cone cells. S-cone cells respond most strongly to short wavelength light (peak near 420-440 nm) and are also known as blue cones. M-cone cells respond most strongly to medium wavelength light (peak near 534-545 nm) and are also known as green cones. L-cone cells respond most strongly to light of long wavelengths (peak near 564-580 nm) and are also known as red cones. The difference in the signals received from the three cone types allows the brain to perceive all possible colors. Exemplary photoreceptor specific promoters include but are not limited to GRX promoter (Gene Card ID: GC05M095752), CRX promoter (Gene Card ID: GC19P047819), CrxdEN promoter, and GnbQ501/3 promoter (see, e.g., FIG. 2).

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Regulatable AAV-CRX Vectors for Treating CRX-Linked Retinopathies in Mouse Models

The present Example utilizes an AAV-mediated gene augmentation general strategy to treat CRX retinopathies caused by various CRX sequence variants. Since CRX is a regulatory protein, the amount of normal CRX introduced to a sick photoreceptor cells is important and directly related to the therapeutic outcome and side effects (toxicity). To address this challenge, three criteria in the rAAV vector design were accomplished: 1) Cell-type specificity: Photoreceptor-specific promoters were used to avoid targeting other cell types; 2) Dose range: Amplifiable low-to-high strength photoreceptor promoters were used; and 3) Overdose toxicity: A tune down/shut-off mechanism incorporated.

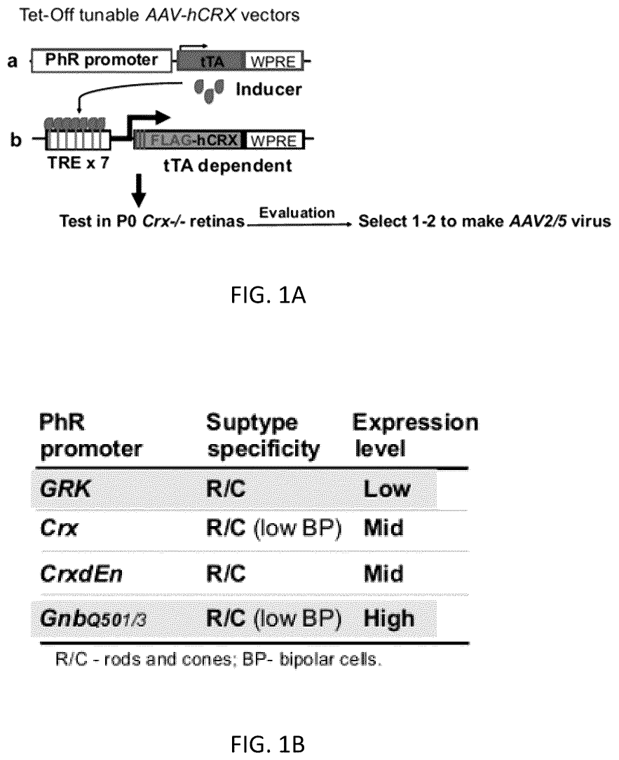

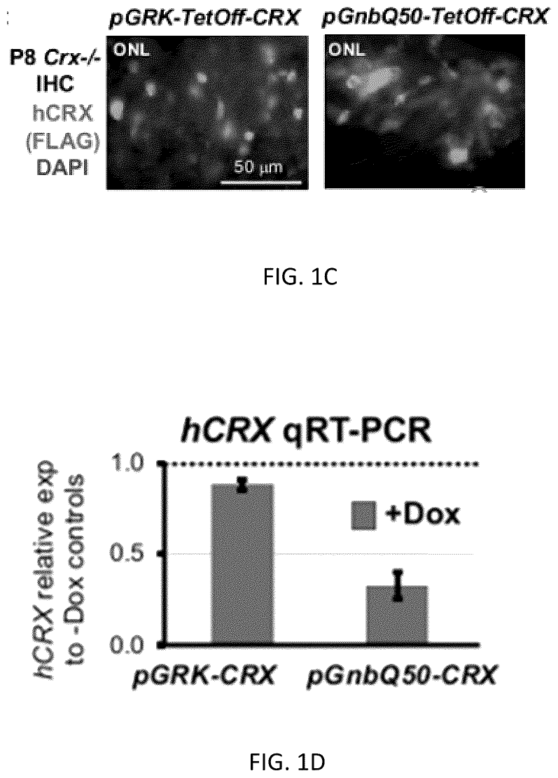

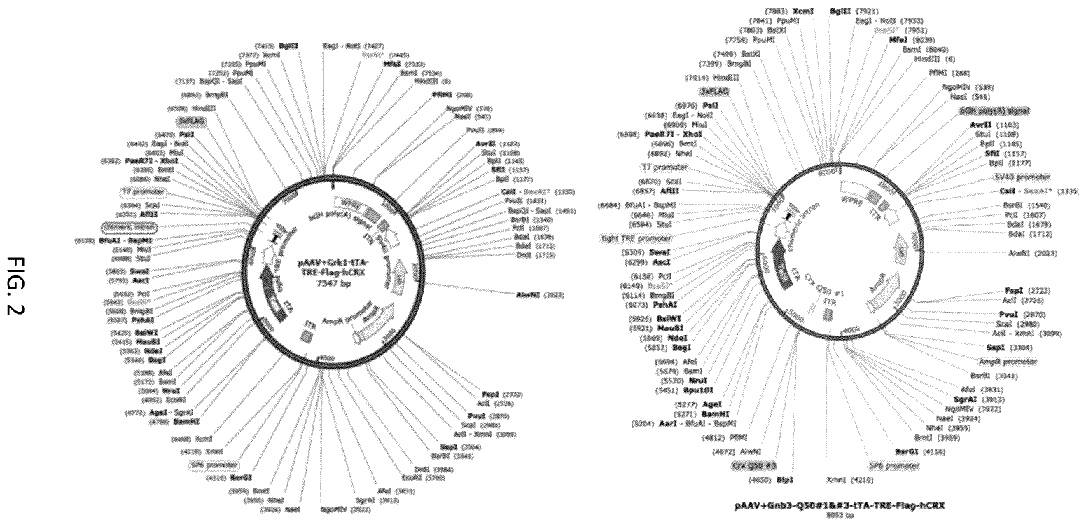

rAAV vector design and testing: Four exemplary photoreceptor promoters that are active in Crx−/− retinae and conserved in the human genome were used (FIG. 1B). Then TetOff regulations was introduced, in which a rTA made by Gene ‘a’ activates Gene ‘b’ (a & b are located in the same vector) to produce FLAG-hCRX (FIG. 1A). This setting ensures sufficient CRX production, as the activity of each promoter is amplified to overcome negative effects of Crx mutations. In an instance where therapeutic CRX is overproduced, the present design allows ectopic CRX production to be lowered by doxycycline (Dox) administration in a dose-dependent manner.

Ex vivo experiments were performed to determine which of the four candidate photoreceptor promoters produces the best dynamic range and cell type specificity for CRX expression. Each construct was electroporated into PO (postnatal day 0) retina explants of Crx−/− mice. After 8 days in culture, CRX expression in the explants was assessed using FLAG immunostaining. Shown in FIG. 1C & FIG. 1D, two such vectors, pGRKTetOff-CRX and pGnbQ50-TetOff-CRX, produced encouraging results: They delivered CRX protein in Crx−/− photoreceptors, and the amount of CRX produced can be tuned down by Dox. Thus, these two vectors were chosen for production of AAV2/5 virus. Ongoing experiments have examined the toxicity and efficacy of the two rAAV2/5-delivered ectopic CRX expression in mouse models of CRX retinopathy.

To test that CRX augmentation is beneficial and can improve the phenotype of diseased photoreceptors, proof-of-concept experiments were carried out in mouse models of CRX disease. These models include: 1) Crx−/− that carries a null mutation in both copies of the Crx gene, modeling recessive Leber congenital amaurosis; and 2) Crx-E168d2/+ that carries a dominant Crx mutation in one copy and normal Crx in the other copy, modeling dominant cone-rod dystrophy. To determine the dose requirement and the treatment age window, a transgenic expression system that carries a Dox-inducible hCRX transgene (TetON-hCRX) was designed and made (FIG. 3). With this system, the expression of the CRX transgene in photoreceptor cells is induced at desired ages by doxycycline supplied in food/water or by intraperitoneal (IP) injections. Phenotype improvements at different stages of disease were then evaluated by measuring changes in 1) target gene expression, 2) photoreceptor subcellular structure, such as the outer segment (OS) formation, and 3) photoreceptor cell survival (ONL thickness).

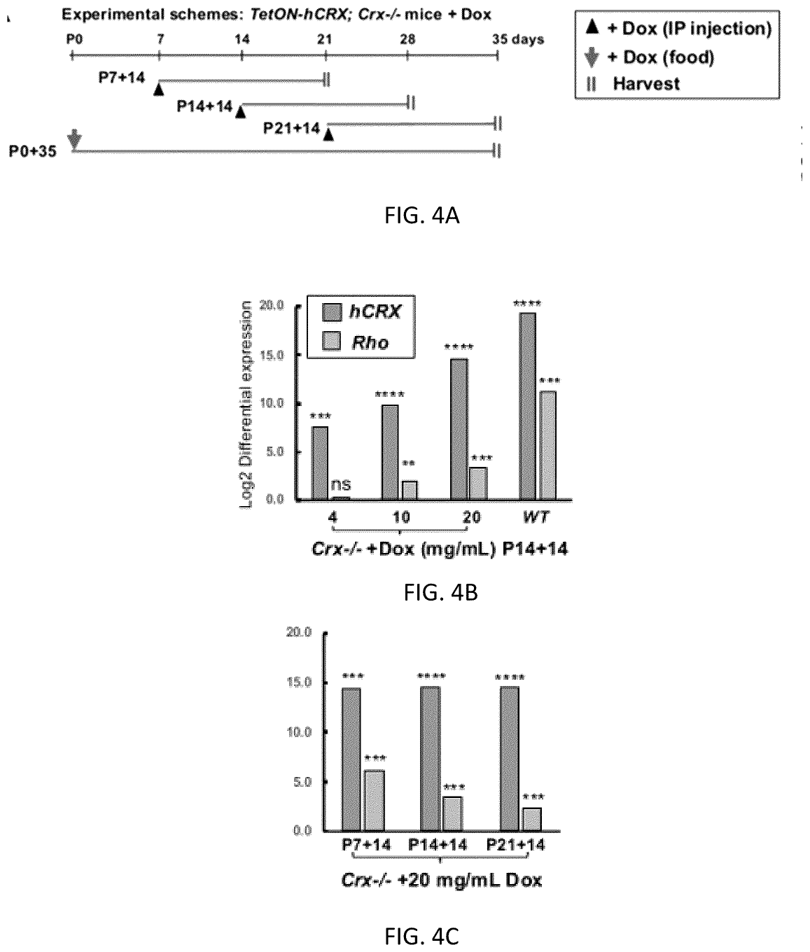

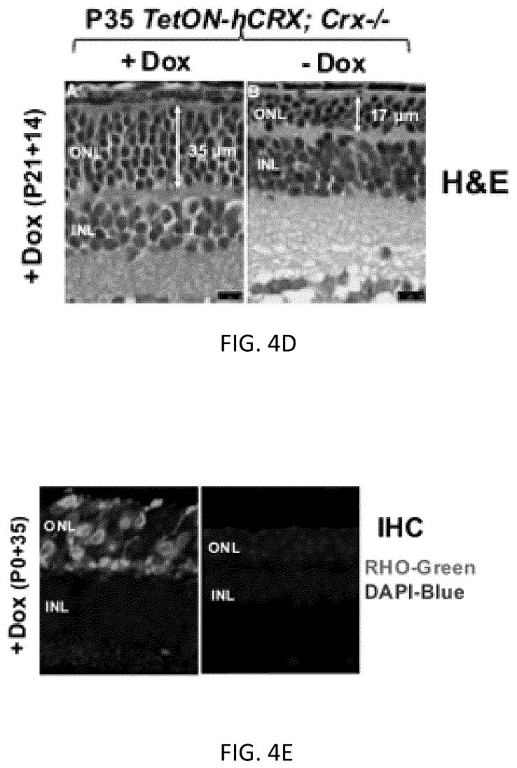

Effects on recessive CRX mutations modeled by Crx−/− mice: all TetOn-hCRX transgenes (with pNrl-Cre) were crossed onto the Crx−/− background. Intraperitoneal (IP) Dox injections (alternate days) or Dox food feeding (200 mg/kg) have yielded reproducible and significant improvement of photoreceptor phenotypes (FIG. 4): 1) hCRX was induced in a [Dox]-dependent manner in Crx−/− retinae at all the ages tested (FIG. 4A-C), confirming the success of the transgene system. 2) Dox-treated mutants displayed significant upregulation of Rho expression which positively correlated with induced hCRX levels. 3) Earlier inductions produced better rescue effects of Rho expression, e.g. P7+14 induction resulted in a 64-fold increase in Rho mRNA as compared to only a 5-fold for P21+14 induction. 4) Signs of improved photoreceptor survival were detected in treated mutants even with a late induction (P21+14, FIG. 4D; and P28+14, data not shown). These results imply Crx−/− photoreceptors maintain a plastic window for therapeutic intervention. 5) Dox food feeding induced Rho expression in rod photoreceptors and improved ONL thickness (FIG. 4E). These results imply Crx−/− photoreceptors maintain a plastic window for therapeutic intervention, demonstrating the feasibility for AAVmediated gene augmentation therapy.

Effects on dominant CRX mutations modeled by Crx-E168d2/+ mice: Could CRX augmentation be a valuable strategy for dominant CRX disease? Dominant CRX mutations cause dominant disease by interfering with the function of normal CRX allele. A Class Ill mouse model E168d2/+ demonstrated that the protein ratio of WT/mutant allele directly correlates with phenotype severity and age of onset. A small increase in the WT/mutant ratio in E168d2neo/+ mice resulted in a much-improved phenotype.

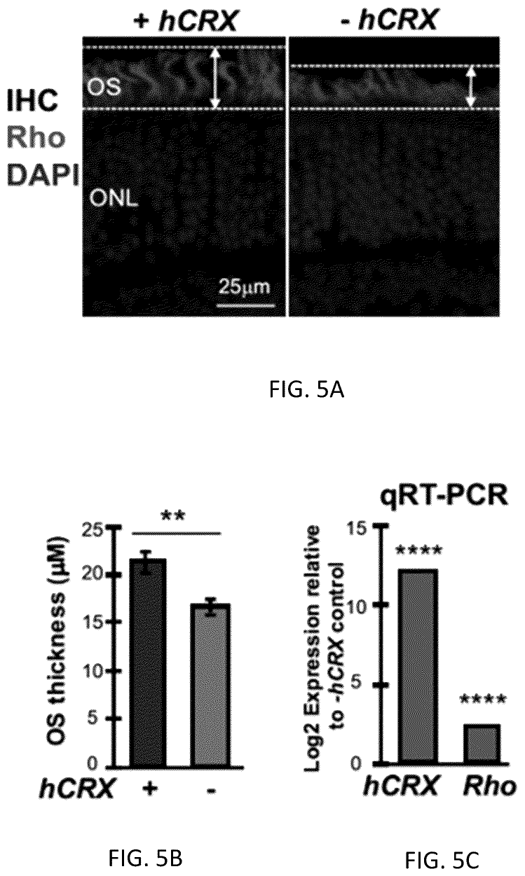

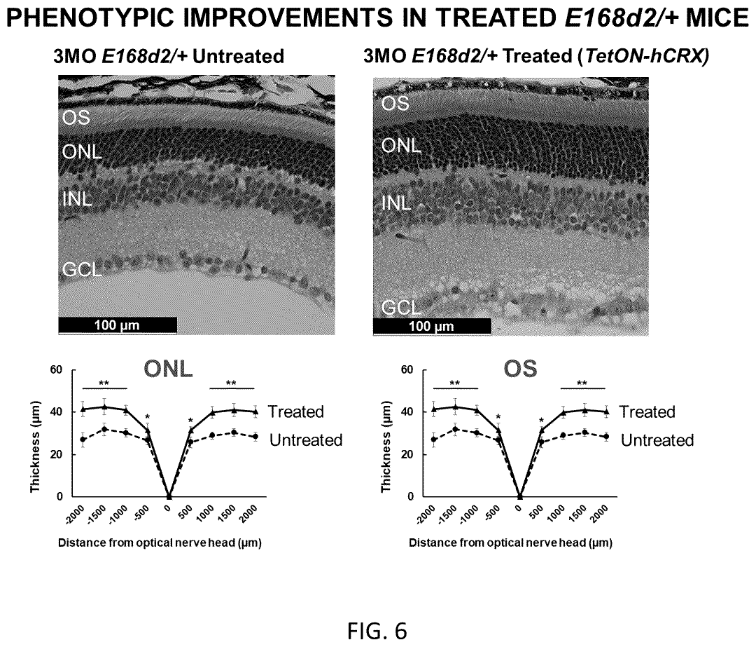

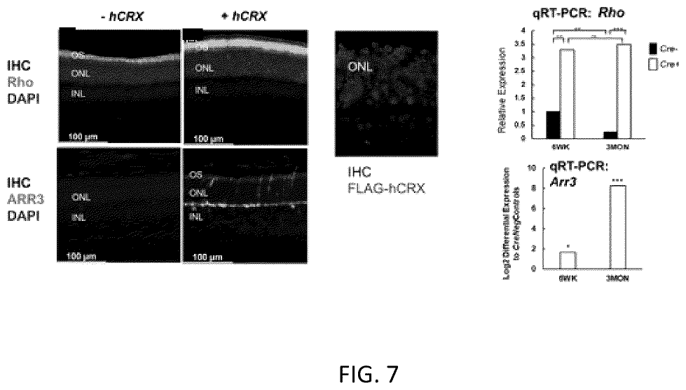

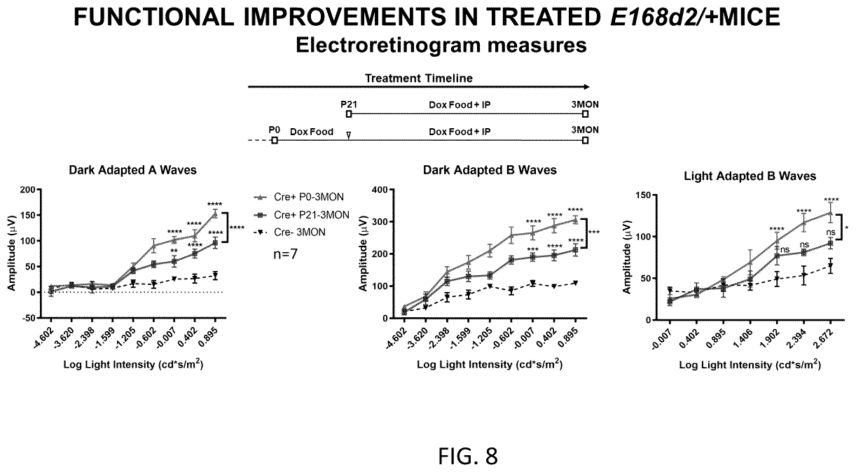

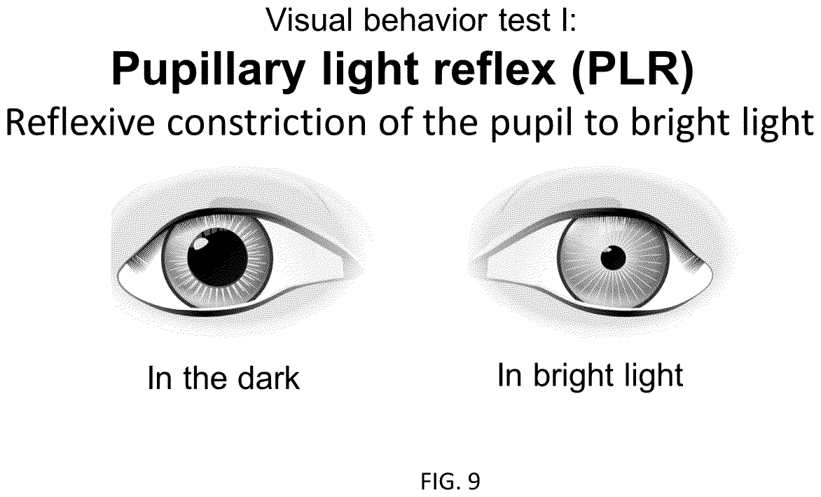

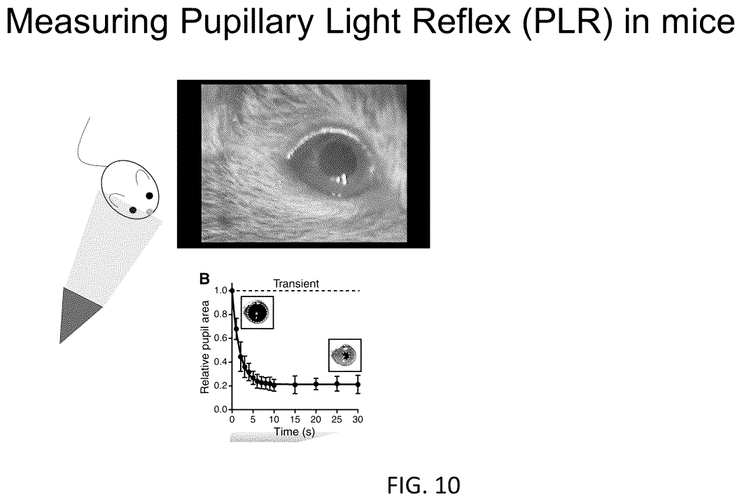

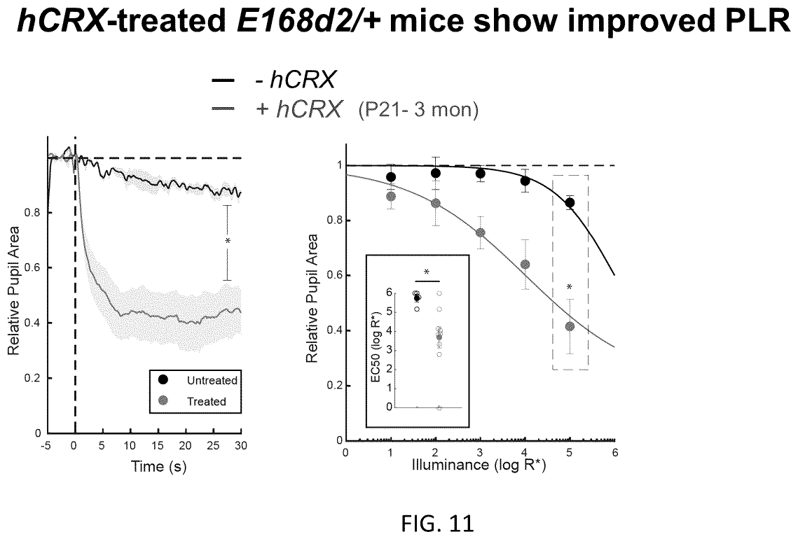

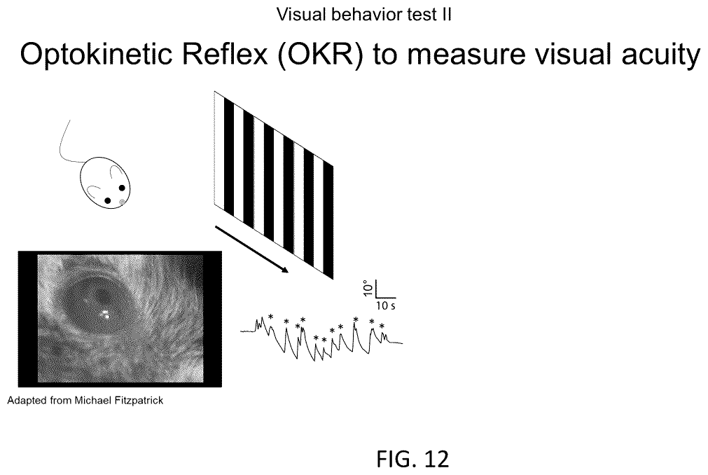

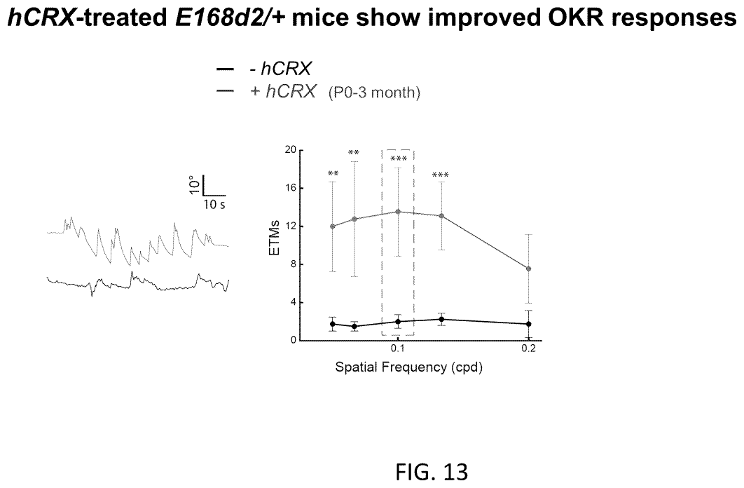

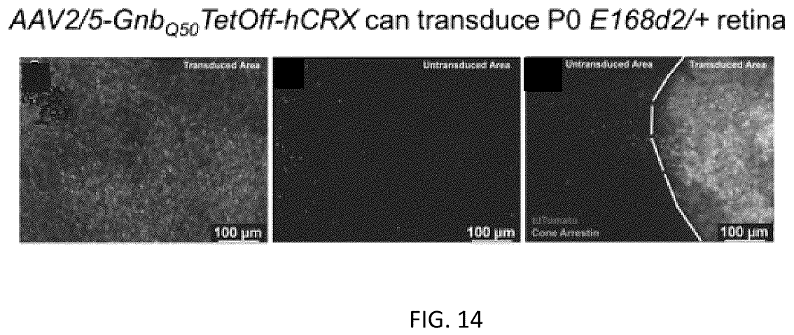

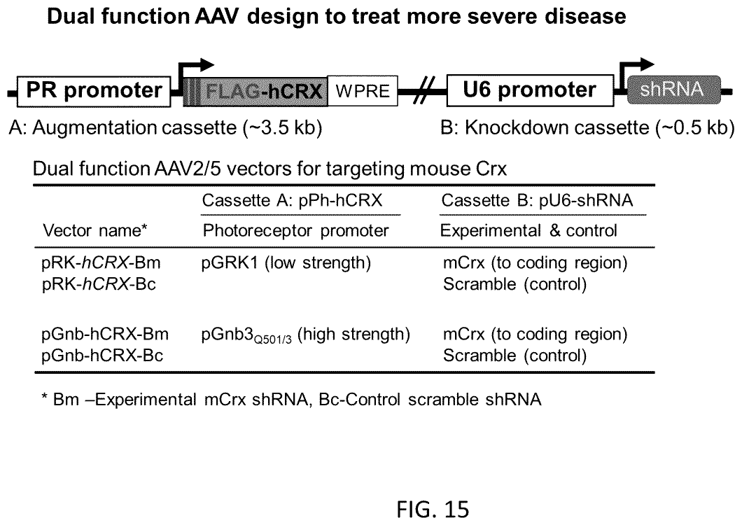

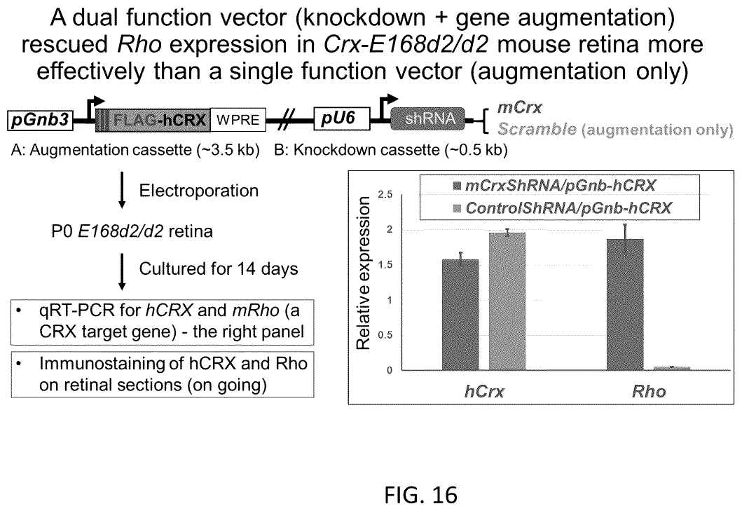

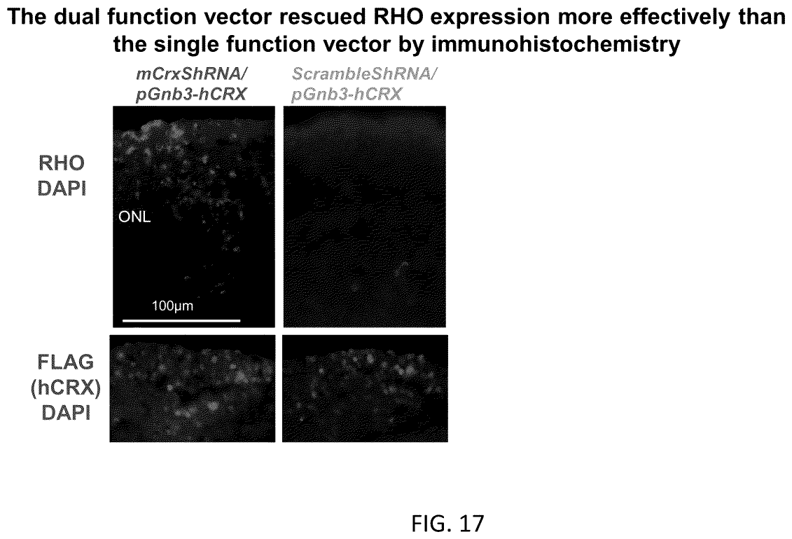

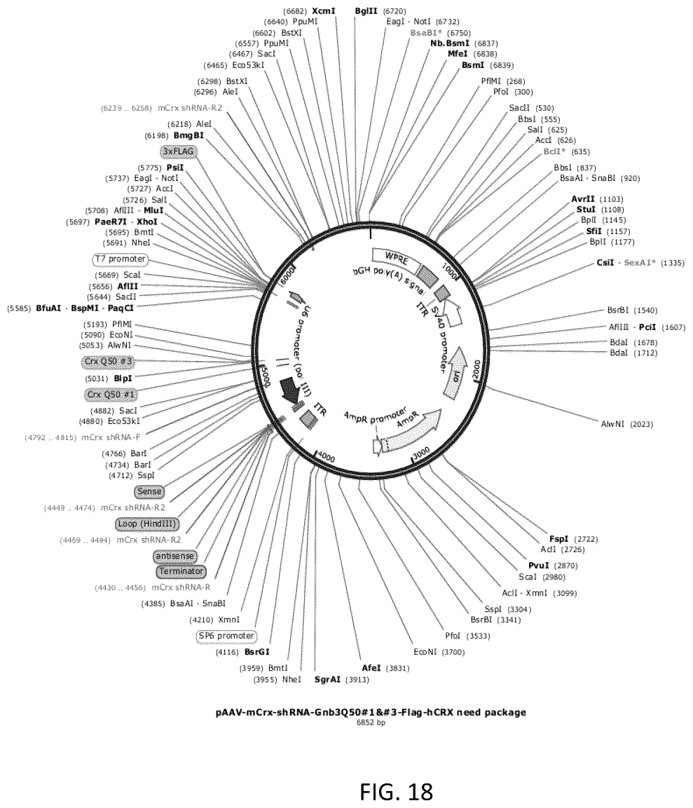

To demonstrate the feasibility of CRX augmentation therapy, the TetOn-hCRX transgene system was crossed to the Crx-E168d2/+ model, in which one copy of the Crx gene carries the E168d2 mutation while the other copy is normal. The transgenic hCRX expression was induced at P21 by feeding the mice with Dox chow (200 mg/kg) and harvested the retina at P35 to access Rhodopsin (Rho) expression and the length of the photoreceptor outer segments (OS). As shown in FIG. 5, with only 14 days of the treatment in young adult mice, Rho expression (A&C) and the OS thickness (A&B) in treated animals (+hCRX) were significantly improved as compared to the untreated control animals (−hCRX). Thus, raising of the WT/mutant ratio by expressing an ectopic normal CRX have beneficial effects on photoreceptor integrity in dominant CRX disease. a small improvement of photoreceptor function in treated animals were also observed when the treatment duration increased to 6 weeks (data not shown). These results set a foundation for development of rAAV-delivered CRX augmentation therapy.

What is claimed is:

1. A regulatable, recombinant adeno-associated viral vector comprising:
a first promoter operably linked to the first nucleic acid sequence, wherein the first nucleic acid sequence encodes a tetracycline-controlled transactivator; and
a second promoter comprising a tet-regulated element (TRE), wherein the second promoter is operably linked to the second nucleic acid sequence, wherein the second nucleic acid sequence encodes a CRX protein that provides a therapeutic effect on a CRX-mediated retinopathy.

2. The regulatable, adeno-associated viral vector of claim 1, wherein the TRE region comprises at least one tetracycline resistance operator binding site and wherein the promoter is a minimal promoter region comprising a TATA box and transcription start site.

3. The regulatable, adeno-associated viral vector of claim 1 or claim 2, wherein the first promoter is a photoreceptor cell specific promoter.

4. The regulatable, adeno-associated viral vector of claim 3, wherein the photoreceptor cell specific promoter is selected from a GRK promoter, a CRK promoter, a CrxdEN promoter and a Gnbq501/3 promoter.

5. The regulatable, adeno-associated viral vector of any one of claims 1-4, wherein the second promoter is regulatable by tetracycline or doxycycline.

6. The regulatable, adeno-associated viral vector of any one of claims 1-5, wherein the second promoter is “OFF” regulatable, wherein the expression is about 5% or less compared to the expression without tetracycline or doxycycline.

7. The regulatable, adeno-associated viral vector of any one of claims 1-6, wherein the promoter system is “OFF” regulatable, wherein the expression is about 1% or less compared to the expression without tetracycline or doxycycline.

8. The regulatable, adeno-associated viral vector of any one of claims 1-7, wherein the vector pAAV.

9. The regulatable, adeno-associated viral vector of any one of claims 1-7, wherein the adeno-associated viral vector is an AAV2 serotype.

10. The regulatable, adeno-associated viral vector of any one of claims 1-7, wherein the adeno-associated viral vector is an AAV5 serotype.

11. The regulatable, adeno-associated viral vector of any one of claims 1-8, wherein the adeno-associated viral vector is an AAV2/5 serotype.

12. A method of delivering a regulatable, adeno-associated viral vector to a photoreceptor cell in a subject with a CRX-mediated retinopathy, the method comprising:
providing a recombinant adeno-associated viral (rAAV) vector, the vector comprising:
a first promoter operably linked to the first nucleic acid sequence, wherein the first nucleic acid sequence encodes a tetracycline-controlled transactivator; and
a second promoter comprising a tet-regulated element (TRE), wherein the second promoter is operably linked to the second nucleic acid sequence, wherein the second nucleic acid sequence encodes a CRX protein; and
administering the rAAV vector to the photoreceptor cell.

13. The method of claim 12, wherein the TRE region comprises at least one tetracycline resistance operator binding site and wherein the promoter is a minimal promoter region comprising a TATA box and transcription start site.

14. The method of claim 12 or claim 13, wherein the first promoter is a photoreceptor cell specific promoter.

15. The method of claim 14, wherein the photoreceptor cell specific promoter is selected from a GRK promoter, a CRK promoter, a CrxdEN promoter and a Gnbq501/3 promoter.

16. The method of any one of claims 12-15, wherein the wherein the second promoter is regulatable by tetracycline or doxycycline.

17. The method of claim 16, wherein the method further comprises administering tetracycline or doxycycline after the administration of the rAAV vector to reduce expression of CRX in the photoreceptor cell.

18. The method of claim 17, wherein the expression of CRX is about 5% or less compared to the expression without tetracycline or doxycycline.

19. A method of treating a subject having or suspected of having a CRX-mediated retinopathy, the method comprising:
providing a recombinant adeno-associated viral (rAAV) vector, the vector comprising:
a first promoter operably linked to the first nucleic acid sequence, wherein the first nucleic acid sequence encodes a tetracycline-controlled transactivator; and
a second promoter comprising a tet-regulated element (TRE), wherein the second promoter is operably linked to the second nucleic acid sequence, wherein the second nucleic acid sequence encodes a CRX protein; and
administering the rAAV vector to the subject.

20. The method of claim 19, wherein the TRE region comprises at least one tetracycline resistance operator binding site and wherein the promoter is a minimal promoter region comprising a TATA box and transcription start site.

21. The method of claim 19 or claim 20, wherein the first promoter is a photoreceptor cell specific promoter.

22. The method of claim 21, wherein the photoreceptor cell specific promoter is selected from a GRK promoter, a CRK promoter, a CrxdEN promoter and a Gnbq501/3 promoter.

23. The method of any one of claims 19-22, wherein the wherein the second promoter is regulatable by tetracycline or doxycycline.

24. The method of claim 23, wherein the method further comprises administering tetracycline or doxycycline to the subject after the administration of the rAAV vector to reduce expression of CRX in a photoreceptor cell of the subject.

25. The method of claim 24, wherein the expression of CRX is about 5% or less compared to the expression without tetracycline or doxycycline.

26. A method of treating a cone rod homeobox transcription factor (CRX) autosomal dominant retinopathy in a subject, the method comprising
administering to the subject an effective amount of a nucleic acid molecule comprising a first photoreceptor cell specific promoter operably linked to a nucleic acid molecule encoding a CRX protein and further comprising a second photoreceptor cell specific promoter operably linked to a nucleic acid molecule encoding a shRNA specific for a mutant CRX mRNA, thereby treating the CRX autosomal dominant retinopathy in the subject.

27. The method of claim 26, wherein the CRX autosomal dominant retinopathy is Leber congenital amaurosis (LCA), retinitis pigmentosa, or cone rod dystrophy.

28. The method of claim 27, wherein the CRX autosomal dominant retinopathy is the LCA.

29. The method of any one of claims 26 to 28, comprising administering to the subject a viral vector comprising the nucleic acid molecule.

30. The method of claim 29, wherein the viral vector is a lentivirus vector or an adeno-associated vims (AAV) vector.

31. The method of claim 30, wherein the viral vector is the AAV vector, and wherein the AAV vector is an AAV2, AAV5, AAV8 virus vector or any combination thereof.

32. The method of claim 31, wherein the AAV vector is AAV2/5.

33. The method of any of claims 26 to 28, comprising administering to the subject a nanoparticle or a dendrimer comprising the nucleic acid molecule.

34. The method of any one of claims 26 to 33, wherein the first promoter is GnbQ50.

35. The method of any one of claims 26 to 34, wherein the second promoter is constitutively active.

36. The method of claim 35, wherein the second promoter is U6 promoter.

37. The method of any one of claims 26 to 36, wherein the nucleic acid molecule is ad ministered intravitreally, subretinally or to the retina of the subject.

38. The method of any one of claims 26 to 37, wherein the subject is a human.

39. The method of any one of claims 26 to 38, wherein the method increases cone Arrestin, Rhodopsin, and/or cone Opsin expression in the retina of the subject.

40. The method of any one of claims 26 to 39, wherein the method includes selecting the subject with the CRX autosomal dominant retinopathy.

41. A composition comprising an effective amount of a nucleic acid molecule comprising a first photoreceptor cell specific promoter operably linked to a nucleic acid molecule encoding a CRX protein and further comprising a second photoreceptor cell specific promoter operably linked to a nucleic acid molecule encoding a shRNA specific for a mutant CRX mRNA for use in treating a cone rod homeobox transcription factor (CRX) autosomal dominant retinopathy in a subject.

42. The composition of claim 41, wherein the CRX autosomal dominant retinopathy is Leber congenital amaurosis (LCA), retinitis pigmentosa, or cone rod dystrophy.

43. The composition of claim 42, wherein the CRX autosomal dominant retinopathy is LCA.

44. The composition of any one of claims 41 to 43, comprising a viral vector, wherein the viral vector comprises the nucleic acid molecule comprising a first photoreceptor cell specific promoter operably linked to a nucleic acid molecule encoding a CRX protein and further comprising a second photoreceptor cell specific promoter operably linked to a nucleic acid molecule encoding a shRNA specific for a mutant CRX mRNA.

45. The composition of claim 44, wherein the viral vector is a lentiviral vector or an adeno-associated vims (AAV) vector.

46. The composition of claim 45, wherein the viral vector is the AAV vector, and wherein the AAV vector is an AAV2, AAV5, AAV8 virus vector or any combination thereof.

47. The composition of claim 46, wherein the AAV vector is AAV2/5.

48. The composition of claim 41, comprising a nanoparticle or a dendrimer, wherein the nanoparticle or dendrimer comprises the nucleic acid molecule.

49. The method of any one of claims 41 to 48, wherein the first promoter is GnbQ50.

50. The method of any one of claims 41 to 49, wherein the second promoter is constitutively active.

51. The method of claim 50, wherein the second promoter is U6 promoter.

52. The composition of any one of claims 41 to 51, formulated for retinal or subretinal administration.

53. The composition of any one of claims 41 to 52, wherein the subject is a human.

54. The composition of any one of claims 41 to 53, wherein treating a CRX autosomal dominant retinopathy comprises increasing cone Arrestin, Rhodopsin, and/or cone Opsin expression in the retina of the subject.

55. The nucleic acid molecule of claim 41.

56. A vector comprising the nucleic acid molecule of claim 55.

57. The vector of claim 56, wherein the vector is a viral vector.

58. The viral vector of claim 57, wherein the viral vector is an AAV vector.

59. A composition comprising an effective amount of a nucleic acid molecule comprising a photoreceptor cell specific promoter operably linked to a nucleic acid molecule encoding a CRX, wherein the promoter is not a CRX promoter or a promoter that is trans activated by wild-type CRX for use in treating a cone rod homeobox transcription factor (CRX) autosomal dominant retinopathy in a subject.

60. The composition of claim 59, wherein the CRX autosomal dominant retinopathy is Leber congenital amaurosis (LCA), retinitis pigmentosa, or cone rod dystrophy.

61. The composition of claim 60, wherein the CRX autosomal dominant retinopathy is LCA.

62. The composition of any one of claims 59 to 61, comprising a viral vector, wherein the viral vector comprises the nucleic acid molecule of claim 59.

63. The composition of claim 62, wherein the viral vector is a lentiviral vector or an adeno-associated virus (AAV) vector.

64. The composition of claim 63, wherein the viral vector is the AAV vector, and wherein the AAV vector is an AAV2, AAV5, AAV8 virus vector or any combination thereof.

65. The composition of claim 64, wherein the AAV vector is AAV2/5.

66. The composition of claim 59, comprising a nanoparticle or a dendrimer, wherein the nanoparticle or dendrimer comprises the nucleic acid molecule.

67. The method of any one of claims 59 to 66, wherein the promoter is GnbQ50.

68. The composition of any one of claims 59 to 67, formulated for retinal or subretinal administration.

69. The composition of any one of claims 59 to 68, wherein the subject is a human.

70. The composition of any one of claims 59 to 69, wherein treating a CRX autosomal dominant retinopathy comprises increasing cone Arrestin, Rhodopsin, and/or cone Opsin expression in the retina of the subject.

71. The nucleic acid molecule of claim 59.

72. A vector comprising the nucleic acid molecule of claim 55.

73. The vector of claim 72, wherein the vector is a viral vector.

74. The viral vector of claim 73, wherein the viral vector is an AAV vector.
